(19) United States
(12) Patent Application Publication
(10) Pub. No.: US 2023/0279338 A1
(43) Pub. Date: Sep. 7, 2023

(54) SELF-DESTRUCTING SALMONELLA AS INNATE IMMUNITY ACTIVATOR TO IMPROVE FOOD SAFETY

(71) Applicant: UNIVERSITY OF FLORIDA RESEARCH FOUNDATION, INCORPORATED, Gainesville, FL (US)

(72) Inventors: Roy CURTISS, Gainesville, FL (US); Vinicius LIMA, Gainesville, FL (US)

(21) Appl. No.: 18/018,938

(22) PCT Filed: Jul. 29, 2021
(86) PCT No.: PCT/US2021/043675

§ 371 Date: Jan. 31, 2023

Related U.S. Application Data
(60) Provisional application No. 63/059,931, filed on Jul. 31, 2020.

(51) Int. Cl. C12N 1/20; C12N 1/36

(52) U.S. Cl. CPC C12N 1/20; C12N 1/36

(57) ABSTRACT

Disclosed is a live self-destructing attenuated adjuvant Salmonella strain, or a derivative thereof, capable of safe in ovo inoculation into embryonated avian eggs without reduction in hatchability. In certain examples, the live self-destructing attenuated adjuvant Salmonella strain comprises an attenuated Salmonella typhimurium (S. typhimurium) bacterium, comprising one or more mutations resulting in in vivo self-destruction selected from the group consisting of Δalr, ΔdadB, ΔasdA, ΔP_{asdA}::TT araC P_{araBAD}asd, ΔP_{dadB}::TT araC P_{araBAD}dadB, ΔP_{asdA}::TT rhaRSP_{rhaBAD}asd, ΔP_{dadB}::TT rhaRSP_{rhaBAD}dadB and/or ΔP_{murA}::TT rhaRSP_{rhaBAD}murA. Also disclosed are method of using the attenuated adjuvant Salmonella strains to inoculate Avian species.

### INTRODUCTION

Prevention of morbidity and mortality from infections of animals and humans with pathogens is desirable. Attainment of this objective would significantly benefit society if more and more infectious diseases were controlled by more effective vaccinations. Unfortunately, vaccines to prevent infections by many pathogens do not exist and many existing vaccines only induce partial protective immunity or require multiple repeat vaccinations to sustain adequate protection. We have observed over many years in using recombinant attenuated Salmonella vaccines (now called Protective Immunity Enhanced Salmonella vaccines (PIESVs)) to deliver protective antigens, that animals receiving an empty vector control strain not delivering a protective antigen invariably gave low levels of protective immunity exceeding the no survival level of animals receiving saline but significantly less than in animals receiving vaccine strains delivering protective antigens (Table 1). This protection has also been observed several months after the last immunization. We have ascribed these results to induction of a potent sustained innate immunity that provides time for some animal hosts to develop an induced acquired immunity as a response to the challenge pathogen. We further studied and improved by further modification the ability of these live self-destructing attenuated Salmonella strains to serve as potent adjuvants. Characterization of these self-destructing attenuated adjuvant Salmonella (SDAAS) strains has been accomplished as we also demonstrated superior effectiveness in enhancing the ability of the BCG vaccine to confer enhanced protective immunity to mice challenged with Mycobacterium tuberculosis H37Rv. The long-term objective is to develop universal SDAAS strains and determine the optimal route(s), dose(s) and time(s) of administration to enhance protection against pathogen challenge in the absence of vaccination and to enhance induction of prolonged acquired immunity by co-administration with vaccines to prevent infectious diseases. We have further directed these endeavors to administer SDAAS strains as live adjuvants by in ovo inoculation of 18-day old chicken embryos to enhance some level of protection of newly hatched chicks from colonization by and disease from exposure during the first days of life to various pathogens.

### OVERVIEW

All successful pathogens have evolved means to either infect the host in a stealth mode to be undetectable and/or suppress, modulate or circumvent induction of immunity and/or synthesize subterfuge antigens that induce immune responses that confer no protective immunity and/or devise means to colonize and persist in the host. To circumvent these problems and the fact that most attenuating mutations reduce immunogenicity (158), we have continuously modified Salmonella vaccine vectors to eliminate these immunosuppressive means as well as to also exhibit in vivo regulated delayed attenuation and regulated delayed protective antigen synthesis (159-162). Since live bacterial vaccines have the potential to persist in the environment if shed, we devised means to achieve regulated delayed lysis so that viable vaccine cells do not persist in vivo or survive if shed into the environment (163). Since these vaccine strains have nearly the same ability as the wild-type virulent parent to invade and colonize internal effector lymphoid tissues in the vaccinated host, they induce superior protective immunity. Based on these attributes and the observation that these PIESV vectors are entirely safe when administered to two-hour old mice or to pregnant mice or to protein-malnourished mice or to immunodeficient SCID mice, the NIH Office of Science Policy (overseeing the Recombinant Advisory Committee) and the UF Biosafety Committee permits use of these strains under level 1 containment and under settings simulating commercial rearing for farm animals and in out-patients for human trials. Since vaccination of animals with these PIESV strains not delivering protective antigens conferred a low but significant level of protection after challenge with bacterial, viral and parasite pathogens (Table 1), we directed efforts to discover how best to render modified Salmonella strains to be superior adjuvants in eliciting innate immune responses. We thus retained properties to lessen Salmonella abilities to suppress, modulate or evade inducing immunity in combination with abilities to undergo in vivo lysis. Thus, while the SDAAS strains have some of the same attributes as PIESV strains, they are designed to lyse very soon after inoculation, do not possess means for regulated delayed attenuation or antigen synthesis since they do not deliver any protective antigens and do not themselves induce high-level immune responses or protective immunity to Salmonella or other pathogens. Their sole purpose is thus to significantly enhance induction of innate immunity by in ovo administration to 18-day old chick embryos. The objective is for these SDAAS strains to provide some level of protection to newly hatched chicks from colonization by and disease caused from exposure during the first days of life to various pathogens frequently infecting and colonizing chickens early in life. It is also expected that such in ovo administration of SDAAS strains should enhance the efficacy and level of protective immunity induced by other vaccines that might be administered to newly hatched chicks at day of hatch or anytime thereafter.

### BRIEF DESCRIPTION OF THE DRAWINGS

FIG. 1. Chicken embryo receiving SDAAS strain in the amniotic fluid by in ovo inoculation.

FIG. 2. Induction of TLR4 and NOD1 signaling by Salmonella strains (χ9052 and χ12499) on HEK-Blue-m-TLR4/mNOD1 cell lines. HEK-Blue cell lines, expressing either mTLR4 or mNOD1, were stimulated with χ9052 and χ12499. TLR4/NOD activation was measured by SEAP activity after incubation of HEK-Blue-mTLR4 (A) and HEK-Blue-mNOD1 (B) cells with χ9052 and χ12499, respectively. LPS was used as a positive control for assays with HEK-Blue-mTLR4 cells.

FIG. 3. Enhanced induction of antibody production by co-administration of Salmonella adjuvant strain. Mice inoculated with 100 μg Ovalbumen s.c. alone and together with either 50 μl Alum s.c. or 5×10⁶ CFU of Family A strain χ9052.

FIG. 4. A. Structure of Salmonella lipid A and components controlled by Salmonella and Francisella genes. B. Structural changes due to expression of F. tularensis IpxE gene.

FIG. 5. Salmonella adjuvant strain has higher abilities to activate TLR4 and TLR5 than either LPS or flagellin. HEK-Blue-mTLR4/TLR5 cells were stimulated with χ12518. TLR activation was measured by SEAP activity after incubation with χ12518. LPS and flagellin (100 ng) were used as positive controls.

FIG. 6. Self-destructing attenuated adjuvant Salmonella (SDAAS) strain enhances protection of mice against M. tuberculosis H37Rv challenge. BCG (5×10⁴ CFU s.c., χ12068 (pYA4891) (5×10⁴ CFU s.c. and 1×10⁷ CFU i.n.) and Family B strain SDAAS χ12518 (5×10⁴ CFU i.v.) were administered to groups of 10 mice on day zero. Mice were challenged with aerosolized ˜50 CFU Mtb H37Rv 5 weeks later and euthanized six weeks later after challenge to determine surviving Mtb cells in lungs and spleens.

FIG. 7. Hatchability of embryonated chicken eggs inoculated at 18 days of incubation with two different SDAAS strains. Inoculation doses (1×10⁶ up to 1×10⁹ CFU) were the same for both strains. SDAAS strains used were Family A strain χ12517 (Δalr-3 ΔdadB4 ΔasdA33 ΔfliC180 ΔpagP81::P_{Ipp} IpxE ΔpagL7 ΔIpxR9) and Family B strain χ12518 (ΔP_{asdA55}::TT araC P_{araBAD} asd Δalr-3 ΔP_{dadB66}::TT araC P_{araBAD} dadB ΔfliC180 ΔpagP81::P_{Ipp} IpxE ΔpagL7ΔIpxR9). This study included 2 control groups, one inoculated with BSG only and another not inoculated.

FIG. 8. Hatchability of embryonated chicken eggs inoculated at 18 days of incubation with four different SDAAS strains. Inoculation doses (1×10⁵, 1×10⁷ and 1×10⁹ CFU) were the same for all strains. SDAAS strains used were Family A strains χ12553 (Δalr-3 ΔdadB4 ΔasdA33 ΔfliC180 Δ(hin-fljBA)-219) and χ12554 (Δalr-3 ΔdadB4 ΔasdA33 ΔfliC180 ΔpagP81::P_{Ipp} IpxE ΔpagL7ΔIpxR9 Δ(hin-fljBA)-219) and Family B strains χ12547 (Δalr-3 ΔP_{dadB66}::TT araC P_{araBAD} dadB ΔP_{asdA55}::TT araC P_{araBAD} asdA ΔfliC180 Δ(hin-fljBA)-219) and χ12548 (Δalr-3 ΔP_{dadB66}::TT araC P_{araBAD} dadB ΔP_{asdA55}::TT araC P_{araBAD} asdA ΔfliC180 ΔpagP81::P_{Ipp} IpxE ΔpagL7ΔIpxR9 Δ(hin-fljBA)-219). The control group wasn't inoculated or had an opening in the eggshell.

FIG. 9. Compiled data of 3 independent hatchability studies. Embryonated chicken eggs were inoculated at 18 days of incubation with two different Family A SDAAS strains. Inoculation doses (1×10⁵ up to 1×10⁹ CFU) were the same for both strains. SDAAS strains used were χ12553 (Δalr-3 ΔdadB4 ΔasdA33 ΔfliC180 Δ(hin-fljBA)-219) and χ12554 (Δalr-3 ΔdadB4 ΔasdA33 ΔfliC180 ΔpagP81::P_{Ipp} IpxE ΔpagL7 ΔIpxR9 Δ(hin-fljBA)-219). All studies included 2 control groups, one inoculated with BSG only and another not inoculated.

FIG. 10. Compiled data of 3 independent hatchability studies. Embryonated chicken eggs were inoculated at 18 days of incubation with two different Family B SDAAS strains. Inoculation doses (1×10⁵ up to 1×10⁹ CFU) were the same for both strains. SDAAS strains used were χ12547 (ΔP_{asdA55}::TT araC P_{BAD} asd Δalr-3 ΔP_{dadB66}::TT araC P_{BAD} dadB ΔfliC180 Δ(hin-fljBA)-219) and χ12548 (ΔP_{asdA55}::TT araC P_{BAD} asd Δalr-3 ΔP_{dadB66}::TT araC P_{BAD} dadB ΔfliC180 ΔpagP81::P_{Ipp} IpxE ΔpagL7ΔIpxR9 Δ(hin-fljBA)-219). All studies included 2 control groups, one inoculated with BSG only and another not inoculated.

FIG. 11. Survival of chicks from eggs inoculated with three different SDAAS strains and challenged at day-of-hatch with APEC strain χ7122. Embryonated eggs were inoculated at 18 days of incubation with 1×10⁹ CFU of strain χ12553 (Δalr-3 ΔdadB4 ΔasdA33 ΔfliC180 Δ(hin-fljBA)-219) and 1×10⁵ CFU of strains χ12547 (ΔP_{asdA55}::TT araC P_{araBAD} asd Δalr-3 ΔP_{dadB66}::TT araC P_{araBAD} dadB ΔfliC180 Δ(hin-fljBA)-219) and χ12548 (ΔP_{asdA55}::TT araC P_{araBAD} asd Δalr-3 ΔP_{dadB66}::TT araC P_{araBAD} dadB ΔfliC180 ΔpagP81::P_{Ipp} IpxE ΔpagL7ΔIpxR9 Δ(hin-fljBA)-219). Chicks were challenged at day-of-hatch with 2×10⁷ CFU of strain χ7122 in the yolk-sac.

FIG. 12. Comparison of two independent hatchability studies. Embryonated chicken eggs were inoculated at 18 days of incubation with six different Family A SDAAS strains. The inoculation dose (1×10⁹ CFU) was the same for all strains. SDAAS strains used were 112606 (Δalr-3 ΔdadB4 ΔasdA33 ΔfliC180 Δ(hin-fljBA)-219 ΔpagP8), χ12625 (χ12606 plus ΔIpxR9), χ12629 (χ12625 plus ΔpagL7), χ12638 (χ12629 plus ΔeptA4), χ12640 (χ12638 plus ΔarnT6) and χ12650 (χ12640 plus ΔsifA26). Both studies included 2 control groups, one inoculated with BSG only and another not inoculated.

FIG. 13. Hatchability study comparing 2 different SDAAS strains. Embryonated chicken eggs were inoculated at 18 days of incubation with two different SDAAS strains. Embryonated eggs were inoculated with 1×10⁸ CFU of Family A strain χ12640 (Δalr-3 ΔdadB4 ΔasdA33 ΔfliC180 Δ(hin-fljBA)-219 ΔpagP8 ΔIpxR9 ΔpagL7 ΔeptA4 ΔarnT6) and 1×10⁵ CFU of Family B strain χ12669 (ΔP_{asdA55}::TT araC P_{araBAD} asd Δalr-3 ΔP_{dadB66}::TT araC P_{araBAD} dadB ΔfliC180 ΔpagP81::P_{Ipp} IpxE ΔpagL7 ΔIpxR9 Δ(hin-fljBA)-219 ΔarnT6 ΔeptA4 ΔsifA26 ΔwbaP45 ΔrecA62) at 18 days of incubation.

FIG. 14. Table 1

### DEFINITIONS

The terms “avian” and “avian subjects” or “bird” and “bird subjects” as used herein, are intended to include males and females of any avian or bird species, and in particular are intended to encompass poultry which are commercially raised for eggs, meat or as pets. Accordingly, the terms “avian” and “avian subject” or “bird” and “bird subject” encompass chickens, turkeys, ducks, geese, quail, pheasant, parakeets, parrots, cockatoos, cockatiels, ostriches, emus and the like. In particular embodiments, the subject is a chicken or a turkey. Commercial poultry includes broilers and layers, which are raised for meat and egg production, respectively. The avian to be innoculated can be an in ovo, live fetus or embryo or may be a hatched bird, including newly-hatched (i.e., about the first one, two or three days after hatch), adolescent, and adult birds.

In particular embodiments, the bird is about six-, five-, four-, three-, two- or one-week of age or less. In other representative embodiments, the avian subject is a naïve subject, i.e., has not previously been exposed to the antigen against which immunity is desired.

The vaccine according to the invention may be prepared and marketed in the form of a suspension or in a lyophilized form and additionally contains a pharmaceutically acceptable carrier or diluent customary for such compositions. Carriers include stabilisers, preservatives and buffers. Suitable stabilisers are, for example SPGA, carbohydrates (such as sorbitol, mannitol, starch, sucrose, dextran, glutamate or glucose), proteins (such as dried milk serum, albumin or casein) or degradation products thereof. Suitable buffers are for example alkali metal phosphates. Suitable preservatives are thimerosal, merthiolate and gentamicin. Diluents include water, aqueous buffer (such as buffered saline) and polyols (such as glycerol).

The terms “effective inoculating amount” or “effective inoculating dose,” as used herein, unless otherwise indicated, means a dose of the adjuvant composition sufficient to induce an innate immune response in the treated birds that is greater than the innate immunity of non-inoculated birds. In the case of birds treated in ovo, an “effective inoculating dose” indicates a dose sufficient of the SDAAS strain to induce an innate immune response in the hatched birds that have been treated in ovo that is greater than the inherent innate immunity of birds that were not inoculated in ovo. An effective inoculating dose in any particular context can be routinely determined using methods known in the art.

An “effective inoculating dose” comprises one dose of the SDAAS composition with sufficient numbers of CFUs so as to achieve the desired level of protection of newly hatched chicks from colonization by and disease from exposure during the first days of life to various pathogens. The individual dose is administered in ovo, usually between 17.5 and 19.2 days of chicken egg incubation but will differ if used for other avian species with differing durations for hatching of embryos.

The term “Innate Immunity” is used here to refer to the natural defenses displayed by an animal host species exposed to a foreign antigen or pathogen and includes natural defense barriers, non-specific phagocytic cells and elicitation of cytokines and chemokines that recruit other cells in the immune system to commence the development of acquired immunity with display of mucosal and systemic antibody and mucosal and internal cellular immunities.

The term “adjuvant” as used here refers to an agent that induces in an inoculated animal host a heightened means to withstand infection and to elicit an improved level of acquired immunity in the host when exposed to an antigen, vaccine or pathogen or part thereof. Adjuvants can also enhance display of natural barriers to infection by pathogens and diminish the ability of pathogens to infect, colonize or cause disease.

The disclosed self-destructing attenuated adjuvant Salmonella strain, or a derivative thereof, may be prepared and marketed in the form of a suspension or in a lyophilized form and additionally contains a pharmaceutically acceptable carrier or diluent customary for such compositions. Carriers include stabilisers, preservatives and buffers. Suitable stabilisers are, for example SPGA, carbohydrates (such as sorbitol, mannitol, starch, sucrose, dextran, glutamate or glucose), proteins (such as dried milk serum, albumin or casein) or degradation products thereof. Suitable buffers are for example alkali metal phosphates. Suitable preservatives are thimerosal, merthuilate and gentamicin. Diluents include water, aqueous buffer (such as buffered saline) and polyols (such as glycerol).

The term “live self-destructing attenuated adjuvant Salmonella strain” refers to a Salmonella strain that possesses one or more mutations that facilitate lysis in vivo (e.g. impairing synthesis of essential constituents of peptidoglycan layer or LPS of the organism), one or more mutations that provide auxotrophy (e.g. dependence on an amino acid or in which synthesis of amino acid is dependent on a sugar); one or more mutations to alter synthesis of flagellar components, one or more mutations to enhance recruitment of innate immunity (e.g. mutations that enhance induction of TLR4, TLR5, TLR8, TLR9, NOD1 and/or NOD2), one or more mutations enhancing DNA degradation in Salmonella cells), and/or one or more mutations that suppress, evade, modulate, eliminate or diminish means of decreasing induction of effective immunogenicity.

The term “derivative” in reference to derivatives of a live self-destructing attenuated adjuvant Salmonella strain refers to descendant cells of a live self-destructing attenuated adjuvant Salmonella strain.

The term “pathogen” as used herein refers to a bacteria, virus, fungus or parasite that is capable of infecting and/or causing adverse symptoms in a subject. Examples of specific pathogens include, but are not limited to, Salmonella spp, Esherichia coli strains, Clostriduim spp, Campylobacter spp, Eimeria spp and to influenza virus (e.g. avian influenza virus).

All microbes including pathogens possess damage-associated molecular patterns (DAMPs) and pathogen associated molecular patterns (PAMPs) (more generally referred to as microbe associated molecular patterns (MAMPs)) that are recognized by pattern recognition receptors on the surface of or internally in host cells to recruit innate immune responses with production of cytokines and chemokines.

The term “administering in ovo” or “in ovo administration,” as used herein, unless otherwise indicated, means administering an adjuvant composition to a bird egg containing a live, developing embryo by any means of penetrating the shell of the egg and introducing the adjuvant composition. Such means of administration include, but are not limited to, in ovo injection of the adjuvant composition.

In certain embodiments, the compositions are administered in the final quarter of egg incubation of the avian subject. Where the subject is a chicken, the final quarter to administer the composition of this invention in ovo would be during the period from day through day 20 of fertile egg incubation, and in particular embodiments, the composition can be administered on day 18 or day 19 of incubation. When the subject is a turkey, the final quarter for administration would be during the period from day 21 through day 28 of incubation and in particular embodiments, the compositions can be administered on day 24 or day 25 of incubation. In other embodiments wherein the subject is a goose, the final quarter of administration would be during the period from day 23 through day 31 of incubation and in particular embodiments, the compositions can be administered on day 28 or day 29 of incubation. In further embodiments wherein the subject is a duck, the final quarter of administration would be during the period from day 21 through day 28 of incubation and in particular embodiments, the compositions can be administered on day 25 or day 26 of incubation.

For other avian species, the final quarter of incubation and thus the optimal range of days for in ovo administration of a composition of this invention can be determined according to methods well known in the art. For example, a muscovy duck has an incubation period in the range of 33-35 days, a ringneck pheasant has an incubation period of 23-24 days, a Japanese quail has an incubation period of 17-18 days, a bobwhite quail has an incubation period of 23 days, a chuckar partridge has an incubation period of 22-23 days, a guinea has an incubation period of 26-28 days and a peafowl has an incubation period of 28 days. The incubation period is affected by the temperature of incubation. This is further varied by the different body temperatures of species and breeds.

In particular embodiments, the composition can comprise, the SDAAS strain in a suitable excipient such as buffered saline. Since the SDAAS strain will be a lyophilized product reconstituted just prior to in ovo inoculation, it might contain products present to aid in the preservation and stability of the SDAAS product during lyophilization.

### DETAILED DESCRIPTION

The present disclosure is based on studies using newly constructed S. typhimurium UK-1 SDAAS strains that display the regulated lysis phenotype and expression of DAMPs and PAMPs. The initial studies were to evaluate safety of these strains in embryonated chicken eggs inoculated at 18 days of incubation to allow maximum hatchability and the determination of how they impact weight gain and feed conversion efficiency of newly hatched chicks. Other studies evaluate the ability of the SDAAS strains to induce an early protective innate immune response to reduce colonization by different Salmonella enterica serotypes, one APEC serotype, C. perfringens, C. jejuni and L. monocytogenes in white leghorn and broiler chicks. Lastly, the nature of the protective response, regarding its duration, cells and immune components involved is characterized.

Accordingly, some embodiments of the present disclosure pertain to a live self-destructing attenuated adjuvant Salmonella strain, or a derivative thereof, capable of safe in ovo inoculation into embryonated avian eggs without reduction in hatchability. The live self-destructing attenuated adjuvant Salmonella strain or derivative thereof, may include an attenuated Salmonella typhimurium (S. typhimurium) bacterium, comprising one or more mutations resulting in in vivo self-destruction selected from the group consisting of Δalr, ΔdadB, ΔasdA, ΔP_{asdA}::TT araC P_{araBAD} asd, ΔP_{dadB}::TT araC P_{araBAD} dadB, ΔP_{asdA}::TT rhaRS P_{rhaBAD} asd, ΔP_{dadB}::TT rhaRS P_{rhaBAD} dadB and/or ΔP_{murA}::TT rhaRS P_{rhaBAD} murA. In a specific embodiment, the live self-destructing attenuated adjuvant Salmonella strain or a derivative of includes the mutations ΔfliC180 and Δ(hin-fljBA to enhance recruitment of innate immunity via interaction with PRR TLR5. In another embodiment, the live self-destructing attenuated adjuvant Salmonella strain or a derivative of, includes the mutations ΔpagP or ΔpagP::P_{Ipp} IpxE, ΔpagL, ΔIpxR, ΔarnT and/or ΔeptA to enhance recruitment of innate immunity via interaction with PRR TLR4. In a further embodiment, the live self-destructing attenuated adjuvant Salmonella strain or a derivative of, includes the mutations ΔwaaC, ΔwaaG, ΔwaaL, ΔwbaP, Δpmi and/or Δrfc to enable improved interaction of bacterial adjuvant surface MAMPs and DAMPs to enhance recruitment of innate immunity via interaction with host PRRs. In yet another specific embodiment, the live self-destructing attenuated adjuvant Salmonella strain or a derivative of, includes the mutations ΔsifA and/or ΔrecA to enhance recruitment of innate immunity via interaction with host cell internal PRRs such as, but not limited to, TLR8, TLR9, NOD1 and/or NOD2.

In another embodiment, a method of inoculating embryonated avian eggs is provided. The method involves administering, in ovo, an effective inoculating amount of a live self-destructing attenuated adjuvant Salmonella strain or derivative thereof disclosed herein or a derivative thereof. Administering the strain or derivative thereof induces innate immunity of hatched offspring from the inoculated embryonated avian eggs. In a further embodiment, administering the strain or derivative thereof does not reduce hatchability of the inoculated embryonated avian eggs. Further still, administering the strain or derivative thereof decreases severity of infection of hatched offspring from the inoculated embryonated avian eggs by avian pathogens. In specific examples, the avian pathogens are E. coli (APEC) strains.

In yest another embodiment, provided is a composition that includes an amount of a live self-destructing attenuated adjuvant Salmonella strain disclosed herein, or derivative thereof, and a carrier and/or diluent.

Overview

After World War II chicken became a popular food item (1) and since then the increased demand for poultry meat and eggs worldwide led to the development and application of new technologies in animal agriculture that resulted in great improvements in how animal protein is produced (2). From 1956, when the average body weight of a broiler at 56 days of age was around two pounds, to today when a modern chicken can easily reach more than 10 pounds at 42 days of age with a much better feed conversion rate. All these advancements also brought many challenges. Birds are now much more susceptible to infection by pathogens and there is a growing concern regarding transmission of drug-resistant pathogens through the food chain (3). This led the government to create new policies to reduce and ultimately eliminate the use of antibiotics during food animal production (4-8). It is well stablished that many enteric pathogens can be transmitted to humans through consumption of poultry meat and eggs (9). Salmonella spp, C. jejuni, E. coli, C. perfringens and L. monocytogenes are all listed as common and important foodborne pathogens by the CDC and there are many available reports linking consumption of contaminated poultry products to disease caused by these agents (10).

Avian Pathogenic E. coli and zoonotic risk to humans. In addition to causing disease in poultry, recent studies have implicated APEC strains as including those responsible for extra-intestinal pathogenic E. coli (ExPEC) infections in humans (11-14). Pathogens in the ExPEC group are characterized into specific pathotypes that are related to the clinical presentation induced in the host and include uropathogenic E. coli (UPEC), neonatal-meningitis E. coli (NMEC) and newborn meningitis (NBM) (15). Several reports have demonstrated that poultry meat can serve as a source of ExPEC, with up to 92.3% of samples testing positive for E. coli and 46.1% of them having virulence factors associated with ExPEC and 15.6% identified as UPEC (16). In 2010 the CDC released a report to inform the public on the zoonotic risk of ExPECs and their possible transmission through poultry meat (17).

Salmonella species are important zoonotic pathogens that cause gastrointestinal disease and systemic disease in humans and animals. Salmonellosis develops different syndromes, including gastroenteritis, enteric fever (typhoid fever), and bacteremia, and as asymptomatic carriage in animals and humans (18). It is the leading cause of foodborne illness in the U.S., with 35% of the hospitalizations and 28% of the deaths (19). There are approximately 1.03 million cases of non-typhoidal Salmonella each year in the U.S., costing an economic loss of approximately $3.31 billion due to premature mortality, disability, and medical and productivity costs, with an annual loss of 16,782 quality-adjusted life years (20). Salmonella has a broad host range and adapts to survive in a wide range of different environments, even up to 16 months in dry feed stored at 25° C. (21, 22). Although a large number of human infections are associated with food animal sources, infections also come from pets, reptiles, fruits, vegetables and other humans (23-26). Transmission of Salmonella to humans typically occurs when ingesting foods that are contaminated by animal feces or cross-contaminated by other sources (27). Among these sources, poultry and poultry-associated products are widely recognized as being among the most important vehicles for human Salmonella infections according to CDC reports (25, 28-34). With increasing consumption of poultry and poultry products, the number of salmonellosis associated with poultry continues to be a significant public health issue in the U.S.

Campylobacter as a major foodborne pathogen. Campylobacter is a leading cause of bacterial foodborne gastroenteritis worldwide and is a major public health problem (35-37). A recent estimate by the CDC indicates that C. jejuni is not only among the most common causes of foodborne illnesses in humans (over 800,000 cases per year), but also is a leading cause of hospitalization (over 8,000 annually) (19). Patients infected with C. jejuni often experience watery/bloody diarrhea, abdominal cramps, nausea, and fever. Severe neurological sequelae, bacteremia and other extra-intestinal complications may develop infrequently (38). C. jejuni is widespread in food-producing animals, especially in poultry. The majority of human C. jejuni infections are predominantly associated with poor handling of raw chicken or consumption of undercooked chicken (39-47). The predominant role of poultry in human campylobacteriosis is supported by high prevalence of C. jejuni in both live birds and on carcasses, findings from epidemiological studies, and detection of identical genotypes in both poultry and human infections (43, 44, 48, 49).

L. monocytogenes contamination of poultry products and the risks to public health. L. monocytogenes is the causative agent of listeriosis and its pathogenesis is mainly associated with consumption of contaminated food products (10, 25, 50-52). The CDC estimates 1,600 infections by Listeria every year. In humans, symptoms are variable but severe cases include septicemia, meningitis and gastroenteritis. Infection frequently requires hospitalization and mortality rates range from 20 to 30%. Immunocompromised, elderly individuals and pregnant woman are the most susceptible to infection. Spontaneous abortion, premature labor and neonatal disease are commonly seen in pregnant woman infected with L. monocytogenes (20, 53). Similar to Salmonella, L. monocytogenes is also classified in serotypes based on cell wall antigens (54). Serotype 11 (1/2b) is commonly found in chicken meat, being among the most frequently reported in human listeriosis (50, 51, 55, 56). Growing evidence shows that live birds can become asymptomatic carriers of L. monocytogenes, with up to 14% of flocks being reported to be contaminated (56). Just recently, 8.5 million pounds of frozen ready-to-eat poultry meat had to be recalled and taken off the market due to possible contamination with Listeria. The United States is the biggest poultry meat producer in the world and this year poultry meat became the most consumed animal protein worldwide. Reduction or complete elimination of L. monocytogenes in poultry products would greatly benefit public health and the poultry industry.

C. perfringens: A reemerging pathogen with zoonotic potential. For many years antibiotics and ionophores were used as growth promoters in commercial farms, but public concern with transmission of antibiotic-resistant bacteria through the food chain resulted in regulations that limited and/or completely eliminated the use of these drugs in food animal production (4, 6, 7). With this, diseases that where once eliminated started to reemerge (57). Necrotic enteritis caused by C. perfringens became the most prevalent disease in broiler production in the last decade and some cases have also been reported in laying hens raised in cage-free systems (58-61). Economic losses are mainly associated with reduced growth efficiency and decreased feed conversion, although cases with up to 50% mortality have been reported (61, 62). In addition to causing necrotic enteritis in poultry, C. perfringens also produce enterotoxins during sporulation which cause foodborne illness in humans. C. perfringens type A causes gastroenteritis and type C produces necrotic enteritis in humans (63). The high prevalence of the pathogen in broilers results in high percentages of contaminated carcasses and outbreaks traced to consumption of chicken have been reported (9, 10). It is estimated that every year 1 million people become infected and develop gastroenteritis due to consumption of food items contaminated with C. perfringens (19, 24). It is estimated that losses due to necrotic enteritis cost the poultry industry $2 billion annually worldwide (59, 62).

Innate immunity activators induce early protection. The innate immune system possesses a multitude of germline encoded pattern recognition receptors (PRRs), e.g. toll-like receptors, nucleotide-binding oligomerization domain [NOD]-like receptors and RIG-1-like receptors, each recognizing different patterns that are associated with bacterial, viral, parasite and fungal infections (64). Activation of these receptors start a pre-programmed cascade of events that result in rapid activation of the innate immune system (65, 66). Modulation of these immune responses have been extensively explored as an alternative to prevent and treat many infectious diseases and cancer (67-72). Hayashy, et al. (73) have shown that pulmonary administration of a phospholipid-conjugated TLR7 ligand protected mice from infection with Bacillus anthracis, Venezuelan equine encephalitis virus and H1 N1 influenza virus. Verthelyi et al. (74) also reported that administration of CpG oligodeoxynucleotides protected normal and SIV-infected macaques from experimental Leishmania infection. Also, day-of-hatch chicks derived from eggs injected at 18 days of incubation with CpG dinucleotide were protected when challenged at day-of-hatch with lethal doses of APEC strains and were partially protected against infection caused by Avian Infectious Laryngotracheitis Virus (75, 76). TLR9 binding to CpG oligonucleotide drives expression of Type-1 interferons through MyD88 signaling that results in recruitment of different immune cells, including macrophages, neutrophils/heterophils and dendritic cells (77). These results show that administration of PPR agonists can be used to induce an early protective innate immune response against bacterial and viral pathogens using agents that will interact and activate components of the innate immune system.

In ovo immunization to produce early protection of newly-hatched-chicks against pathogens. In 1982 Sharma and Burmester (78) showed that in ovo vaccination of 18-day old chicken embryos was an efficient method to protect day-of-hatch chicks against Marek's disease and that it produced better results when day-of-hatch chicks derived from injected eggs and immunized at day-of-hatch were challenged at 3 days of age with a virulent Marek's Disease Virus. In 1993, the development of modern equipment capable of immunizing thousands of eggs per hour allowed this strategy to be used commercially at a very low cost. Today it is estimated that 20 billion eggs are immunized every year in the United States (79). Since this initial experiment in 1982, many researchers have used this technique to deliver amino acids, antibiotics and more recently, probiotics and components capable of stimulating the immune system (75, 76, 80-83).

Preharvest control strategies for enteric pathogens of zoonotic risk in poultry. Traditionally, antibiotics have been used to treat bacterial infections and for growth promotion in food animals (84). However, this contributed to increasing rates of antibiotic resistance, resulting in contamination of flocks and food products by antibiotic-resistant Campylobacter, Salmonella, Enterococcus, C. perfringens, E. coli and L. monocytogenes thereby increasing risks of human infections (85, 86). Public concerns over the spread of antibiotic resistance in zoonotic bacterial pathogens, which poses a threat to the effectiveness of existing antibiotic therapy in both clinical medical and veterinary practice (87-91), led the European Union in 1999, to ban use of most antibiotics for growth promotion to preserve the effectiveness of important human drugs (92). In 2004, the U.S. FDA banned enrofloxacin in food animals on the grounds that its use contributed to fluoroquinolone resistance in human pathogens. More recently, FDA and the animal food-producing industry have agreed to cease use of growth promotion antimicrobials. However, there are concerns that reductions in antibiotic use in animal production may lead to an increase in foodborne pathogens on meat and other foods of animal origin. It is therefore necessary to develop other effective ways to mitigate emergence of antibiotic-resistant bacteria and to control foodborne pathogens.

Intervention to control foodborne pathogens in poultry. Since contaminated poultry products are the major source of human foodborne pathogen infection (9, 24, 37, 41), early protection of chickens against colonization will be an important strategy to reduce the levels of Salmonella, ExPEC, C. perfringens, C. jejuni and L. monocytogenes in poultry flocks, which will ultimately lead to lower rates of human foodborne illness (93). In general, the on-farm control strategies used to reduce the incidence of enteric pathogens in poultry that can be transmitted through the food chain can be broadly divided into two approaches: 1) prevention of flock colonization by use of biosecurity-based interventions, and 2) prevention and/or reduction of colonization by non-biosecurity based measures such as vaccination, addition of bacteriocins, bacteriophages, feed additives, and competitive exclusion (93-98). Improving biosecurity on farms apparently has a noticeable effect on lowering the overall flock prevalence. However, even the most stringent biosecurity measures do not always have a consistent and predictable effect on controlling these pathogens and their effectiveness on flock prevalence is difficult to assess under commercial settings (94, 99-102). In addition, stringent biosecurity measures are cost-prohibitive, hard to maintain, and their effectiveness seems to vary with production systems (94, 103). Currently the main approach to control Salmonella infection in poultry flocks is the use of vaccines. There are 3 types available, live attenuated, inactivated and subunit; only the former two are licensed for chickens. Currently administered Salmonella vaccines may take from one to two weeks to produce an immune response that is restricted to a few serotypes and allow for infection of animals in the first days of life. Variable efficacy, persistence, reversion to virulence and lack of cross protection are concerns (104-107). Killed vaccines are safe, but they must be delivered by costly injection in birds and require adjuvants to increase efficacy (105-107). There are several E. coli vaccines currently available, including passive and active immunizations, use of inactivated and live vaccines, recombinant and subunit vaccines and immunization against specific virulence factors. Although many options are currently available, no vaccine has proved to be highly efficacious for multiple serotypes in the field. This is why broilers are rarely vaccinated against APEC (108). Currently there are only two licensed vaccine against C. perfringens and no available vaccines against C. jejuni and L. monocytogenes.

We recently discovered a potential solution to these problems during our studies to develop Protective Immunity Enhanced Salmonella Vaccine (PIESV) vectors to deliver protective antigens to induce immunity to a diversity of bacterial, viral and parasite pathogens. In recent years our group developed new and effective strategies to successfully deliver protective antigens to both humans and animals using S. typhimurium as a vaccine carrier (109-111). In these studies, we observed that our PIESV vectors have intrinsic adjuvant properties and induced some level of protection in animals immunized with an empty vector and later challenged with different pathogens (Table 1). We believe that this is due to activation and recruitment of the innate immune system. Recombinant Salmonella strains expressing the regulated delayed lysis phenotype are able to successfully colonize and invade the intestinal mucosa and lymphoid tissues, such as the mucosa associated lymphoid tissue (MALT), gut associated lymphoid tissues (GALT) and spleen (110, 112). These strains have an arabinose-inducible promoter that regulates the expression of gene products that are essential for synthesis and maintenance of the bacterial cell wall, and in their absence the vector will lyse and release peptidoglycan, DNA, RNA, ATP, and other pathogen associated molecular patterns (PAMPs) and damage associated molecular patterns (DAMPs), which have already been extensively shown to activate the innate immune system through interaction with pattern recognition receptors (PRRs) (72, 77, 110).

Results of our previous studies led us to design and construct novel strains, termed Self-Destructing Attenuated Adjuvant Salmonella (SDAAS) strains. These strains have unique attributes that will allow maximum interaction with different components of the innate immune system to produce an early, non-specific, protective response. Based on evidence collected by our group and others (67-72), we hypothesize that injection of embryonated eggs at 18 days of incubation using an SDAAS strain will induce an early protective innate immune response against many bacterial, viral and parasite pathogens, yet allow similar or improved hatchability when compared with non-inoculated eggs.

TABLE 1
Empty vector PIESVs and protective immunity in mice and chickens

| | | | Number | Percent Survival/Protection | | |
|---|---|---|---|---|---|---|
| | | Pathogen | of | | Empty | PIESV + |
| Pathogen | PIESV | Challenge | Experiment | BSG | Vector | Antigen |
| Influenza | PIESV-Flu | Influenza WSN | 3 | 16 | 29 | 90 |
| Y. pestis | PIESV-Yp | Y. pestis (s.c.) | 3 | 0 | 38 | 83 |
| S. pneumoniae | PIESV-Sp | S. pneumoniae | 7 | 0 | 5 | 61 |

| | | | | |
|---|---|---|---|---|
| M. tuberculosis | PIESV-Mtb | M. tuberculosis | 3 | Empty vector control reduced |
| | | | | Mtb colonization more than |
| | | | | BSG |
| C. perfringens | PIESV-Cp | C. perfringens | 4 | Empty vector reduced lesion & |
| | | | | mortality, enhance feed |
| | | | | conversion & weight gain more |
| | | | | than BSG |
| E. tenella | PIESV-Et | E. tenella | 2 | Empty vector enhanced feed |
| | | | | conversion & weight gain more |
| | | | | than BSG |

### EXPERIMENTAL APPROACH

Note that PCT Publication WO/2020/096994 A1 ('994 pub) and PCT/US21/30077 provide extensive background support on SDAAS strains and protocols for their use and implementation as adjuvants. The '994 pub and PCT/US21/30077 are incorporated herein in their entirety to the extent not inconsistent with the teachings herein.

### Example 1. General Materials and Methods

a. Bacterial strains, media and bacterial growth. All previously constructed SDAAS strains for testing in day-of-hatch chicks were derived from the highly virulent S. typhimurium strain UK-1 (146). We previously learned that an attenuated S. typhimurium UK-1 strain will induce protective immunity to challenge with all S. typhimurium strains whereas other S. typhimurium strains attenuated with the same mutations often cannot induce protective immunity to other S. typhimurium strains and definitely not to the highly virulent UK-1 strain (147, Sanapala et al. 2018). LB broth and agar are used as complex media for propagation and plating of Salmonella and APEC strains. SDAAS strains are cultured in LB broth and agar enriched with 50 pg/ml of L-alanine and 50 pg/ml of diaminopimelic acid (Family A strains) or 0.1% L-arabinose and 0.1% rhamnose (if needed) (Family B strains). Brilliant green agar with 15 μg/ml of novobiocin or Salmonella-Shigella agar with 15 μg/ml of nalidixic acid and 15 μg/ml of rifampicin, when necessary, are used to enumerate bacteria in chickens and their tissues. Tetrathionate or selenite broth, with or without supplements, is used to enrich for Salmonella from cecal and intestinal contents, the bursa of Fabricius, liver and spleen. Bacterial growth is monitored spectrophotometrically and by plating for colony counts. Sequenced and well-characterized C. jejuni strain RM221 isolated from chickens is used in challenge studies. This strain is cultured microaerophilically (85% N2, 10% C02, 5% 02) on Mueller-Hinton (MH) medium at 42° C. for 24 h. For C. jejuni isolation from chicken feces and organs, charcoal cefoperazone deoxycholate agar (mCCDA) is used. Cooked meat broth and fluid thioglycolate is used for C. perfringens growth, Tryptose Sulfite Cycloserine Agar (TSC) with egg yolk is used for bacterial titer determination in small intestine samples. C. perfringens is cultured at 37° C. under an anaerobic atmosphere. L. monocytogenes strain L4951 (1/2b) is cultured in brain heart infusion broth and bacterial titers in intestinal and internal organ samples are determined using PALCAM Listeria Agar. Bacterial strains for the challenge studies are listed in Table 2. We also constructed a derivative of the S. typhimurium UK-1 parent χ3761 resistant to both nalidixic acid and rifampicin χ12599 to facilitate quantitative recovery from chickens after challenge infections.

Except for APEC strains and high doses of S. typhimurium (but only in newly hatched chicks) all other enteric pathogens do not cause severe disease in chickens. Nevertheless, they colonize chickens efficiently and can contaminate food products that result in transmission of these enteric pathogens through the food chain to humans who are much more susceptible to infection and disease than are chickens. To evaluate beneficial activities of in ovo administered SDAAS strains one must establish a dose of each pathogen sufficient to colonize 80 to 90 percent of newly hatched chicks and then use a challenge dose 10- and 100-times this dose to evaluate the degree of protection afforded by in ovo SDAAS strain administration. Thus, the doses and possibly the routes of challenge infection need to be established. This can be accomplished by evaluation of colonization frequencies after administering varying doses in CFU of the challenge strains.

TABLE 2
Strains used for challenge studies.

| Strain | Genotype or Phenotype | Reference |
|---|---|---|
| C. perfringens CP4P | Wild Type A, NetB [+] | (148) |
| S. Typhimurium χ3761 | Wild Type B, 1, 4, [5], 12:i:1, 2 | (149) |
| APEC χ7122 | Wild Type O78:K80:H9 | (150) |
| C. jejuni RM1221 | Chicken isolate HS:53 | (151, 152) |
| L. monocytogenes | Wild Type (1/2b) | N/A |
| L4951 | | |

The SDAAS strains used for initial in ovo inoculation and for the derivation of new SDAAS strains are listed in Table 3. The newly constructed strains are listed in Table 5 for Family A strains and Table 6 for Family B strains.

TABLE 3
Parent SDAAS strains used for initial in ovo inoculation (Example 5).

| SDAAS | |
|---|---|
| Strain | Genotype or Phenotype |
| χ12517 | Δalr-3 ΔdadB4 ΔasdA33 ΔfliC180 ΔpagP81::P_{Ipp} IpxE ΔpagL7 ΔIpxR9 |
| χ12518 | Δalr-3 ΔP_{dadB66}::TT araC P_{araBAD} dadB ΔP_{asdA55}::TT araC P_{araBAD} asdA |
| χ12553 | ΔfliC180 ΔpagP81::P_{Ipp} IpxE ΔpagL7 ΔIpxR9 |
| | Δalr-3 ΔdadB4 ΔasdA33 ΔfliC180 Δ(hin-fljBA)-219 |
| χ12554 | Δalr-3 ΔdadB4 ΔasdA33 ΔfliC180 Δ(hin-fljBA)-219 ΔpagP81::P_{Ipp} IpxE |
| | ΔpagL7 ΔIpxR9 Δ(hin-fljBA)-219 |
| χ12547 | Δalr-3 ΔP_{dadB66}::TT araC P_{araBAD} dadB ΔP_{asdA55}::TT araC P_{araBAD} asdA |
| | ΔfliC180 Δ(hin-fljBA)-219 |
| χ12548 | Δalr-3 ΔP_{dadB66}::TT araC P_{araBAD} dadB ΔP_{asdA55}::TT araC P_{araBAD} asdA |
| | ΔfliC180 Δ(hin-fljBA)-219 ΔpagP81::P_{Ipp} IpxE ΔpagL7 ΔIpxR9 |

b. Molecular and genetic procedures. Methods for DNA isolation, restriction enzyme digestion, DNA cloning and use of PCR and real-time PCR for construction and verification of bacterial strains and vectors are standard (45) and methods for generating mutant strains are described in previous publications (46-51).
c. Cell culture methods and use of HEK293 cells to monitor initiation of innate immune responses. We used HEK293 cells with the murine TLR2, TLR4, TLR5, TLR8, TLR9, NOD1 and NOD2 with the NF-κB SEAP reporter system to enable read outs at A650 nm (62,63). SDAAS strains are grown to maximize invasiveness and used to determine cell attachment to, invasion into and survival in Int-407, RAW264.7 and HEK cells. NF-κB production by various MOIs of SDAAS to HEK cells over a 24 h period is measured (64,65).
d. Monitoring immune responses. Changes in gene expression of different chicken cytokines are measured using RT-PCR techniques previously described (156). Flow cytometry is used to determine what types of cells are recruited in this early innate immune response (157).
e. Animal experimentation. Testing of SDAAS strains: Studies on safety of SDAAS strains are conducted in 6- to-8 week old BALB/c mice with SDAAS strains administered at increasing doses and by various mucosal (oral and intranasal) and parenteral (intravenous, intramuscular and subcutaneous) routes. Safety and efficacy studies in chickens are evaluated in SPAFAS white leghorn and commercial broiler embryonated eggs and chicks hatched at UF (see Section IV below). Fertilized eggs are incubated in GQFMFG—Sportsman 1502 incubators and candled every week thereafter until day 18 of incubation. At this time, the eggs are inoculated with 5 different doses (1×10⁵ CFU up to 1×10⁹ CFU) of candidate SDAAS strains suspended in 20 μl of sterile buffered saline with 1% gelatin (BSG). Two control groups are also used, one with embryonated eggs inoculated with BSG only and another group not inoculated. After in ovo injection, the eggs are transferred to a different incubator with appropriate temperature and humidity, where they remain until hatching (day 21). In initial studies comparing hatchability between eggs inoculated with different doses of SDAAS strains and control groups, chicks are euthanized in up to 6 hours after hatch following the new AVMA guidelines for animal euthanasia. After determination of the best SDAAS strain and dose used in initial studies, chicks derived from inoculated eggs are challenged 6 hours after hatch with the strains listed in Table 2 and at doses and routes of inoculation described above. Food and water are provided ad libitum 30 minutes after challenge. SDAAS strains to be evaluated (as well as Salmonella and APEC challenge strains) are grown in LB broth to an OD600 of ˜0.9, sedimented by centrifugation at room temperatures and suspended in PBS at densities of 5×10¹⁰ CFU/ml and decimal dilutions are performed to allow proper strain doses to be administered in 20 μl into eggs and chicks. Procedures similar to those used to grow Salmonella and APEC strains are used to culture the proposed L. monocytogenes strain, the only difference will be that LB broth is replaced by BHI broth. C. perfringens CP4 is grown for 24 h in cooked meat broth and newly hatched chicks are inoculated with 100 μl at 6 and 18 h after hatch by oral gavage. Blood is collected by wing vein puncture to characterize the innate immune response induced by SDAAS strains.

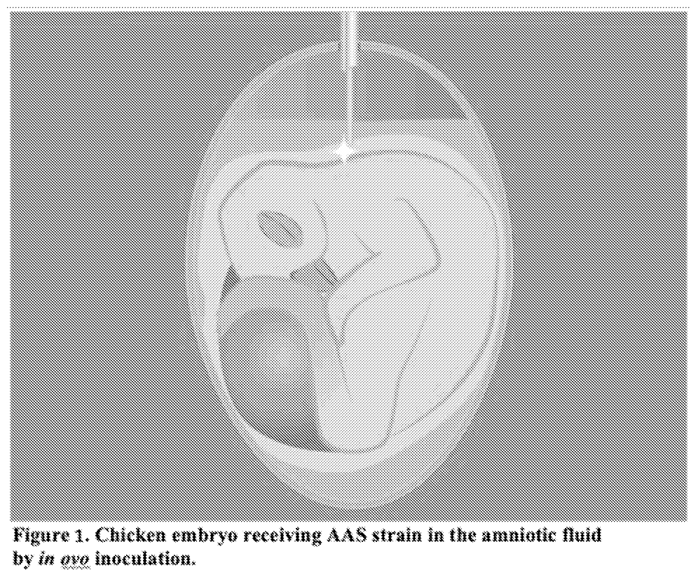

FIG. 1 depicts an 18-day old chicken embryo within an egg being inoculated in ovo with a needle penetrating the eggshell and delivering the inoculated SDAAS strain. Depending on the depth of penetration of the needle, the SDAAS strain can be delivered into the air sac, the allantoic fluid or the amniotic fluid. Inoculation into the amniotic fluid is preferred although comparative evaluation of all three sites will be investigated to determine which location induces the best level of innate immunity while being completely safe and causing no reduction in hatchability.

SDAAS strains are evaluated for induction of early protective innate immune responses that diminish tissue (bursa, liver and spleen) and cecal titers of Salmonella serotype challenge strains, cecal titers of the C. jejuni challenge strain, small intestine titers of the C. perfringens challenge strain, intestine titers of the L. monocytogenes strain and mortality of chicks challenged with the APEC strain. Animals are housed in poultry isolators from hatch until euthanasia. All experimental work is conducted in compliance with the regulations and policies of the Animal Welfare Act and the Public Health Service Policy on Humane Care and Use of Laboratory Animals and approved by the UF IACUC.

f. Statistical analyses and scientific rigor. All studies are repeated and results independently corroborated. Results are analyzed using the most appropriate statistical test from the SAS program to evaluate the relative significance or lack thereof of results obtained. In specific cases, we will consult with the staff at the Clinical Translational Science Institute at UFL who provide help with experimental design and statistical analyses services for animal and human studies and trials.

### Example 2. Mutations and Associated Phenotypes in S. typhimurium SDAAS and PIESV Strains

Table 4 lists all the deletion and deletion-insertion mutations included in SDAAS strains evaluated for contributions to enhance induction of innate immune responses by in ovo inoculation of 18-day old chick embryos. Also included are mutations present in PIESV strains used in some evaluations of SDAAS strain effectiveness.

TABLE 4
Mutations and associated phenotypes in S. Typhimurium adjuvant and vaccine strains^{a}

| Genotype | Phenotype |
|---|---|
| ΔasdA | encodes aspartate semialdehyde dehydrogenase essential for |
| | synthesis of diaminopimelic acid (DAP) necessary for |
| | peptidoglycan synthesis (113) |
| ΔP_{asdA}::TT araC P_{araBAD} asdA | makes synthesis of AsdA dependent on presence of |
| | arabinose |
| ΔasdA::TT araC P_{araBAD} | c2 inactivates asdA and makes synthesis if C2 repressor |
| | dependent on arabinose (114, 115) |
| Δalr and ΔdadB | encodes the two alanine racemases essential for synthesis of D- |
| | alanine necessary for peptidoglycan synthesis (116) |
| ΔP_{dadB66}::TT araC P_{araBAD} dadB | makes synthesis of DadB dependent on presence of |
| | arabinose |
| ΔP_{murA}::TT araC P_{araBAD} murA | makes synthesis of MurA, the first enzyme in the |
| | synthesis of muramic acid, dependent on arabinose in growth |
| | medium and ceases synthesis in vivo due to absence of |
| | arabinose (110, 111) |
| ΔpagP | encodes addition of an additional acyl chain on lipid A that is |
| | immunosuppressive (117) |
| ΔpagP::P_{Ipp} IpxE mutation | causes regulated delayed in vivo synthesis of the codon- |
| | optimized IpxE gene from Francisella tularensis to cause |
| | synthesis of the non-toxic adjuvant form of LPS lipid A |
| | monophosphoryl lipid A (MPLA) (117) |
| ΔpagL and ΔIpxR | eliminates two means by which Salmonella alters LPS |
| | components in vivo to decrease recruitment of innate immunity |
| | by interaction with TLR4 (118) |
| ΔeptA | adds ethanolamine to lipid A (119, 120) |
| ΔarnT | adds 4-amino-4-deoxy-L-arabinose (L-Ara4N) groups to lipid A |
| | (121) |
| ΔfliC180 | specifies a truncated FliC protein containing TLR5 recognition |
| | domain and CD4 epitope(122) |
| Δ(hin-fljBA) | locks in expression of phase I FliC flagellin and precludes |
| | synthesis of phase II FljB flagellin (123-129) |
| ΔwaaC | encodes enzyme need for LPS core synthesis and assembly |
| | (131) |
| ΔwaaG | encodes enzyme need for LPS core synthesis and assembly |
| | (131) |
| ΔwaaL | encodes enzyme that couples LPS O-antigen to LPS core (131) |
| ΔwaaL & ΔpagL::TT araC P_{araBAD} | waaL makes synthesis of the WaaL enzyme that |
| | couples O-antigen to the LPS core synthesis (131)dependent on |
| | presence of arabinose |
| ΔwbaP | encodes an enzyme needed to couple LPS O-antigen to the LPS |
| | core (131) |
| Δpmi | eliminates phosphomannose isomerase that precludes synthesis |
| | of GDP-mannose that is needed for LPS O-antigen synthesis |
| | (130) |
| Δrfc | encodes enzyme necessary for coupling of subsequent LPS O- |
| | antigen subunits onto growing LPS O-antigen chain (130) |
| Δ(wza-wcaM) | eliminates 20 genes encoding enzymes needed for synthesis of |
| | colonic acid, LPS capsular antigen and other polysaccharides to |
| | facilitate lysis, enhance immunogenicity and inhibit biofilm |
| | formation (132, 133) |
| ΔrelA | uncouples growth regulation from a dependence on protein |
| | synthesis (134, 135) |
| ΔrelA::araC P_{BAD} lacI TT | makes synthesis of LacI that represses gene expression |
| | Controlled by P_{trc} dependent on presence of arabinose (109) |
| ΔrecA | necessary for genetic recombination and absence causes |
| | recombination to be lethal with degradation of DNA (Willetts et al. |
| | 1969) |
| ΔrecF | reduces inter- and intra-plasmidic recombination (136-139) |
| ΔsifA | enables Salmonella to escape from the SCV to enter the cytosol |
| | (140, 141) |

^{a}Δ = deletion; TT = transcription terminator; P = promoter

### Example 3. Design, Construction and Characterization of Self-Destructing Attenuated Adjuvant Salmonella (SDAAS) Strains

Based on the results described above, we modified S. typhimurium to develop very safe, highly efficacious strains to use as live adjuvants to rapidly enhance the induced immune responses to a diversity of co-administered vaccines. We thus evaluated 100s of strains with single and multiple combinations of deletion and deletion-insertion mutations (see Table 4) that alter or regulate synthesis and secretion of flagellin, LPS core, O-antigen and lipid A structures, timing and location of lysis (due to mutations controlling D-alanine and diaminopimelic acid (DAP) synthesis) in vivo, and invasiveness with the objective to develop a superior live adjuvant strain that would exhibit complete safety in newborn, pregnant, malnourished and immunodeficient mice. Initially we started with strains that would either commence to lyse immediately after administration (Family A strains derived from χ9052 Δalr-3 ΔdadB4 ΔasdA33) or would undergo 2 to 4 cell divisions in vivo prior to lysis (Family B strains derived from χ12499 Δalr-3 ΔP_{dadB66}::TT araC P_{araBAD} dadB ΔP_{asdA55}::TT araC P_{araBAD} asdA). The Family A and B strains with genotypes and their derivations are listed in Tables 5 and 6, respectively.

TABLE 5
Family A strain genotypes and derivations (most rapidly lysing in vivo

χ9052 Δalr-3 ΔdadB4 ΔasdA33 (parent from which other Family A strains are derived)
χ12503: Δalr-3 ΔdadB4 ΔasdA33 ΔfliC180 from χ9052
χ12512: Δalr-3 ΔdadB4 ΔasdA33 ΔfliC180 ΔpagP81::P_{Ipp} IpxE from χ12503
χ12515: Δalr-3 ΔdadB4 ΔasdA33 ΔfliC180 ΔpagP81::P_{Ipp} IpxE ΔpagL7 from χ12512
χ12517 Δalr-3 ΔdadB4 ΔasdA33 ΔfliC180 ΔpagP81::P_{Ipp} IpxE ΔpagL7 ΔIpxR9 from
χ12515
χ12553: Δalr-3 ΔdadB4 ΔasdA33 ΔfliC180 Δ(hin-fljBA)-219 from χ12503
χ12554: Δalr-3 ΔdadB4 ΔasdA33 ΔfliC180 ΔpagP81::P_{Ipp} IpxE ΔpagL7 ΔIpxR9 Δ(hin-
fljBA)-219 from χ12517
χ12555: Δalr-3 ΔdadB4 ΔasdA33 ΔfliC180 ΔpagP81::P_{Ipp} IpxE ΔpagL7 ΔwaaC41 from
χ12515
χ12556: Δalr-3 ΔdadB4 ΔasdA33 ΔfliC180 ΔpagP81::P_{Ipp} IpxE ΔpagL7 ΔwaaG42 from
χ12515
χ12557: Δalr-3 ΔdadB4 ΔasdA33 ΔfliC180 ΔpagP81::P_{Ipp} IpxE ΔpagL7 ΔwaaL46 from
χ12515
χ12558: Δalr-3 ΔdadB4 ΔasdA33 ΔfliC180 ΔpagP81::P_{Ipp} IpxE ΔpagL7 ΔIpxR9 ΔwaaC41
from χ12517
χ12559: Δalr-3 ΔdadB4 ΔasdA33 ΔfliC180 ΔpagP81::P_{Ipp} IpxE ΔpagL7 ΔIpxR9 ΔwaaG42
from χ12517
χ12560: Δalr-3 ΔdadB4 ΔasdA33 ΔfliC180 ΔpagP81::P_{Ipp} IpxE ΔpagL7 ΔIpxR9 ΔwaaL46
from χ12517
χ12565 Δalr-3 ΔdadB4 ΔasdA33 ΔfliC180 ΔpagP81::P_{Ipp} IpxE ΔpagL7 ΔIpxR9 ΔrecA62
from χ12517
χ12606: Δalr-3 ΔdadB4 ΔasdA33 ΔfliC180 Δ(hin-fljBA)-219 ΔpagP8 from χ12553
χ12608: Δalr-3 ΔdadB4 ΔasdA33 ΔfliC180 ΔpagP81::P_{Ipp} IpxE ΔpagL7 ΔIpxR9 ΔsifA26
from χ12517
χ12625: Δalr-3 ΔdadB4 ΔasdA33 ΔfliC180 Δ(hin-fljBA)-219 ΔpagP8 ΔIpxR9 from χ12606
χ12629: Δalr-3 ΔdadB4 ΔasdA33 ΔfliC180 Δ(hin-fljBA)-219 ΔpagP8 ΔIpxR9 ΔpagL7
from χ12625
χ12638: Δalr-3 ΔdadB4 ΔasdA33 ΔfliC180 Δ(hin-fljBA)-219 ΔpagP8 ΔIpxR9 ΔpagL7
ΔeptA4 from χ12629
χ12640: Δalr-3 ΔdadB4 ΔasdA33 ΔfliC180 Δ(hin-fljBA)-219 ΔpagP8 ΔIpxR9 ΔpagL7
ΔeptA4 ΔarnT6 from χ12638
χ12650: Δalr-3 ΔdadB4 ΔasdA33 ΔfliC180 Δ(hin-fljBA)-219 ΔpagP8 ΔIpxR9 ΔpagL7
ΔeptA4 ΔarnT6 ΔsifA26 from χ12640
χ12661: Δalr-3 ΔdadB4 ΔasdA33 ΔfliC180 Δ(hin-fljBA)-219 ΔpagP8 ΔIpxR9 ΔpagL7
ΔeptA4 ΔarnT6 ΔsifA26 ΔrecA62 (from χ12650)

TABLE 6
Family B strain genotypes and derivations (commencing
to lyse after 2 to 4 cell divisions in vivo).

χ12499 Δalr-3 ΔP_{dadB66}::TT araC P_{araBAD} dadB ΔP_{asdA55}::TT araC P_{araBAD} asdA (parent
from which other Family B strains are derived)
χ12504 Δalr-3 ΔP_{dadB66}::TT araC P_{araBAD} dadB ΔP_{asdA55}::TT araC P_{araBAD} asdA ΔfliC180
from χ12499
χ12513: Δalr-3 ΔP_{dadB66}::TT araC P_{araBAD} dadB ΔP_{asdA55}::TT araC P_{araBAD} asdA ΔfliC180
ΔpagP81::P_{Ipp} IpxE from χ12504
χ12516: Δalr-3 ΔP_{dadB66}::TT araC P_{araBAD} dadB ΔP_{asdA55}::TT araC P_{araBAD} asdA ΔfliC180
ΔpagP81::P_{Ipp} IpxE ΔpagL7 from χ12513
χ12518 Δalr-3 ΔP_{dadB66}::TT araC P_{araBAD} dadB ΔP_{asdA55}::TT araC P_{araBAD} asdA ΔfliC180
ΔpagP81::P_{Ipp} IpxE ΔpagL7 ΔIpxR9 from χ12516
χ12542: Δalr-3 ΔP_{dadB66}::TT araC P_{araBAD} dadB ΔP_{asdA55}::TT araC P_{araBAD} asdA ΔfliC180
ΔpagP81::P_{Ipp} IpxE ΔpagL7 ΔIpxR9 ΔwaaC41 from χ12518
χ12543: Δalr-3 ΔP_{dadB66}::TT araC P_{araBAD} dadB ΔP_{asdA55}::TT araC P_{araBAD} asdA ΔfliC180
ΔpagP81::P_{Ipp} IpxE ΔpagL7 ΔIpxR9 ΔwaaG42 from χ12518
χ12544 Δalr-3 ΔP_{dadB66}::TT araC P_{araBAD} dadB ΔP_{asdA55}::TT araC P_{araBAD} asdA ΔfliC180
ΔpagP81::P_{Ipp} IpxE ΔpagL7 ΔIpxR9 ΔwaaL46 from χ12518
χ12545 Δalr-3 ΔP_{dadB66}::TT araC P_{araBAD} dadB ΔP_{asdA55}::TT araC P_{araBAD} asdA ΔfliC180
ΔwaaC41 from χ12504
χ12546 Δalr-3 ΔP_{dadB66}::TT araC P_{araBAD} dadB ΔP_{asdA55}::TT araC P_{araBAD} asdA ΔfliC180
ΔwaaG42 from χ12504
χ12547 Δalr-3 ΔP_{dadB66}::TT araC P_{araBAD} dadB ΔP_{asdA55}::TT araC P_{araBAD} asdA ΔfliC180
Δ(hin-fljBA)-219 from χ12504
χ12548: Δalr-3 ΔP_{dadB66}::TT araC P_{araBAD} dadB ΔP_{asdA55}::TT araC P_{araBAD} asdA ΔfliC180
ΔpagP81::P_{Ipp} IpxE ΔpagL7 ΔIpxR9 Δ(hin-fljBA)-219 from χ12518
χ12549 Δalr-3 ΔP_{dadB66}::TT araC P_{araBAD} dadB ΔP_{asdA55}::TT araC P_{araBAD} asdA ΔfliC180
ΔwaaL46 from χ12504
χ12564 Δalr-3 ΔP_{dadB66}::TT araC P_{araBAD} dadB ΔP_{asdA55}::TT araC P_{araBAD} asdA ΔfliC180
ΔrecA62 from χ12504
χ12566 Δalr-3 ΔP_{dadB66}::TT araC P_{araBAD} dadB ΔP_{asdA55}::TT araC P_{araBAD} asdA ΔfliC180
ΔpagP81::P_{Ipp} IpxE ΔpagL7 ΔIpxR9 ΔrecA62 from χ12518
χ12567 Δalr-3 ΔP_{dadB66}::TT araC P_{araBAD} dadB ΔP_{asdA55}::TT araC P_{araBAD} asdA ΔfliC180
ΔpagP81::P_{Ipp} IpxE ΔpagL7 ΔIpxR9 ΔwaaL46 ΔrecA62 from χ12544
χ12568 Δalr-3 ΔP_{dadB66}::TT araC P_{araBAD} dadB ΔP_{asdA55}::TT araC P_{araBAD} asdA ΔfliC180
ΔwaaL46 ΔrecA62 from χ12549
χ12570 Δalr-3 ΔP_{dadB66}::TT araC P_{araBAD} dadB ΔP_{asdA55}::TT araC P_{araBAD} asdA ΔfliC180
ΔpagP81::P_{Ipp} IpxE ΔpagL7 ΔIpxR9 ΔarnT6 from χ12518
χ12571: Δalr-3 ΔP_{dadB66}::TT araC P_{araBAD} dadB ΔP_{asdA55}::TT araC P_{araBAD} asdA ΔfliC180
ΔpagP81::P_{Ipp} IpxE ΔpagL7 ΔIpxR9 Δ(hin-fljBA)-219 ΔarnT6 from χ12548
χ12583 Δalr-3 ΔP_{dadB66}::TT araC P_{araBAD} dadB ΔP_{asdA55}::TT araC P_{araBAD} asdA ΔfliC180
ΔpagP81::P_{Ipp} IpxE ΔpagL7 ΔIpxR9 ΔeptA4 from χ12518
χ12584: Δalr-3 ΔP_{dadB66}::TT araC P_{araBAD} dadB ΔP_{asdA55}::TT araC P_{araBAD} asdA ΔfliC180
ΔpagP81::P_{Ipp} IpxE ΔpagL7 ΔIpxR9 Δ(hin-fljBA)-219 ΔeptA4 from χ12548
χ12585 Δalr-3 ΔP_{dadB66}::TT araC P_{araBAD} dadB ΔP_{asdA55}::TT araC P_{araBAD} asdA ΔfliC180
ΔpagP81::P_{Ipp} IpxE ΔpagL7 ΔIpxR9 Δ(hin-fljBA)-219 ΔarnT6 ΔeptA4 from χ12571
χ12586: Δalr-3 ΔP_{dadB66}::TT araC ParaBAD dadB ΔP_{asdA55}::TT araC P_{araBAD} asdA ΔfliC180
ΔpagP81::P_{Ipp} IpxE ΔpagL7 ΔIpxR9 ΔarnT6 ΔeptA4 from χ12570
χ12603 Δalr-3 ΔP_{dadB66}::TT araC P_{araBAD} dadB ΔP_{asdA55}::TT araC P_{araBAD} asdA ΔfliC180
Δ(hin-fljBA)-219 ΔpagP8 (from χ12547)
χ12604: Δalr-3 ΔP_{dadB66}::TT araC P_{araBAD} dadB ΔP_{asdA55}::TT araC P_{araBAD} asdA ΔfliC180
ΔpagP8 ΔpagL7 ΔIpxR9 Δ(hin-fljBA)-219 (from χ12548)
χ12605 Δalr-3 ΔP_{dadB66}::TT araC P_{araBAD} dadB ΔP_{asdA55}::TT araC P_{araBAD}asdA ΔfliC180
Δ(hin-fljBA)-219 ΔpagP8 ΔpagL7 (from χ12603)
χ12609: ΔP_{asdA55}::TT araC P_{araBAD} asd Δalr-3 ΔP_{dadB66}::TT araC P_{araBAD} dadB ΔfliC180
ΔpagP81::P_{Ipp} IpxE ΔpagL7 ΔIpxR9 ΔsifA26 from χ12518
χ12610: ΔP_{asdA55}::TT araC P_{araBAD} asd Δalr-3 ΔP_{dadB66}::TT araC P_{araBAD} dadB ΔfliC180
ΔpagP8 ΔpagL7 ΔIpxR9 Δ(hin-fljBA)-219 ΔsifA26 from χ12604
χ12611: ΔP_{asdA55}::TT araC P_{araBAD} asd Δalr-3 ΔP_{dadB66}::TT araC P_{araBAD} dadB ΔfliC180
ΔsifA26 from χ12504
χ12612: ΔP_{asdA55}::TT araC P_{araBAD} asd Δalr-3 ΔP_{dadB66}::TT araC P_{araBAD} dadB ΔfliC180
ΔpagP81::P_{Ipp} IpxE ΔpagL7 ΔIpxR9 Δ(hin-fljBA)-219 ΔarnT6 ΔeptA4 ΔsifA26 from
χ12585
χ12620: ΔP_{asdA55}::TT araC P_{araBAD} asd Δalr-3 ΔP_{dadB66}::TT araC P_{araBAD} dadB ΔfliC180
ΔpagP81::P_{Ipp} IpxE ΔpagL7 ΔIpxR9 Δ(hin-fljBA)-219 ΔarnT6 ΔeptA4 ΔsifA26 Δpmi-2426
from χ12612
χ12621 : ΔP_{asdA55}::TT araC P_{araBAD} asd Δalr-3 ΔP_{dadB66}::TT araC P_{araBAD} dadB ΔfliC180
ΔpagP81::P_{Ipp} IpxE ΔpagL7 ΔIpxR9 Δ(hin-fljBA)-219 ΔarnT6 ΔeptA4 ΔsifA26 Δrfc-112
from χ12612
χ12623: Δalr-3 ΔP_{dadB66}::TT araC P_{araBAD} dadB ΔP_{asdA55}::TT araC P_{araBAD} asdA ΔfliC180
ΔpagP8 ΔpagL7 ΔIpxR9 Δ(hin-fljBA)-219 ΔeptA4 from χ12604
χ12626: ΔP_{asdA55}::TT araC P_{araBAD} asd Δalr-3 ΔP_{dadB66}::TT araC P_{araBAD} dadB ΔfliC180
ΔpagP81::P_{Ipp} IpxE ΔpagL7 ΔIpxR9 Δ(hin-fljBA)-219 ΔarnT6 ΔeptA4 ΔsifA26 ΔwbΔP45
from χ12612
χ12639: Δalr-3 ΔP_{dadB66}::TT araC P_{araBAD} dadB ΔP_{asdA55}::TT araC P_{araBAD} asdA ΔfliC180
ΔpagP8 ΔpagL7 ΔIpxR9 Δ(hin-fljBA)-219 ΔeptA4 ΔarnT6 from χ12623
χ12641: ΔP_{asdA55}::TT araC P_{araBAD} asd Δalr-3 ΔP_{dadB66}::TT araC P_{araBAD} dadB ΔfliC180
ΔpagP81::P_{Ipp} IpxE ΔpagL7 ΔIpxR9 Δ(hin-fljBA)-219 ΔarnT6 ΔeptA4 ΔsifA26 ΔwaaL46
from χ12612
χ12648: ΔP_{asdA55}::TT araC P_{araBAD} asd Δalr-3 ΔP_{dadB}::TT rhaRS P_{rhaBAD1} dadB ΔfliC180
ΔpagP81::P_{Ipp} IpxE ΔpagL7 ΔIpxR9 Δ(hin-fljBA)-219 ΔarnT6 ΔeptA4 ΔsifA26 from
χ12612
χ12649: ΔP_{asdA55}::TT araC P_{araBAD} asd Δalr-3 ΔP_{dadB}::TT rhaRS P_{rhaBAD1} dadB ΔfliC180
ΔpagP81::P_{Ipp} IpxE ΔpagL7 ΔIpxR9 Δ(hin-fljBA)-219 ΔarnT6 ΔeptA4 ΔsifA26 ΔwbaP45
from χ12626
χ12668: ΔP_{asdA55}::TT araC P_{araBAD} asd Δalr-3 ΔP_{dadB66}::TT araC P_{araBAD} dadB ΔfliC180
ΔpagP81::P_{Ipp} IpxE ΔpagL7 ΔIpxR9 Δ(hin-fljBA)-219 ΔarnT6 ΔeptA4 ΔsifA26 ΔrecA62
from χ12612
χ12669: ΔP_{asdA55}::TT araC P_{araBAD} asd Δalr-3 ΔP_{dadB66}::TT araC P_{araBAD} dadB ΔfliC180
ΔpagP81::P_{Ipp} IpxE ΔpagL7 ΔIpxR9 Δ(hin-fljBA)-219 ΔarnT6 ΔeptA4 ΔsifA26 ΔwbaP45
ΔrecA62 from χ12626

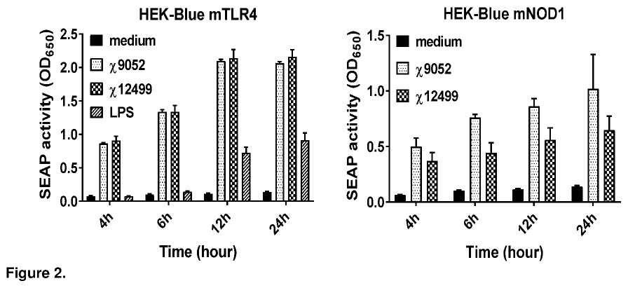

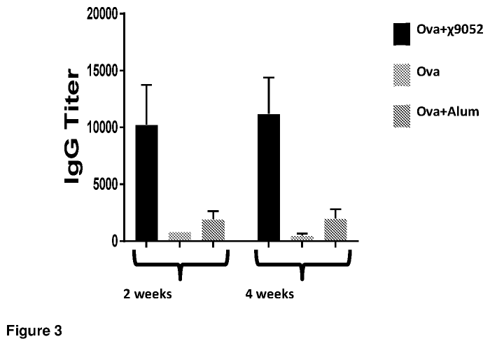

In these comparative evaluative studies with these Self-Destructing Attenuated Adjuvant Salmonella (SDAAS) strains, we made extensive use of additional strains with single and combinations of mutations to fully evaluate their properties in display of agonists and absence of antagonists of Pattern Recognition Receptors (PRRs). We thus used HEK293 cells displaying PRRs on their surface (TLR4 and TLR5) and internally (TLR8, TLR9, Nod1 and Nod2) to evaluate strains for activation of NF-kB that intern initiates the expression of genes to enable display of innate immunity. FIG. 2 is an example of such studies that are fully presented and described in WO 2020/096994 A1 and PCT/US21/30077 as are studies to stimulate synthesis of antibodies to Ova as depicted in FIG. 3.

In regard to synthesis and secretion of flagellin to maximize recruitment of TLR5, we used the ΔfliC180 mutation that specifies a central deletion of the fliC gene eliminating 180 aa of the FliC flagellin but which retains the TLR5 interacting domain and the CD4 epitope. This mutation in conjunction with the phase-lock mutation Δ(hin-fljBA)-219 eliminates motility and synthesis of the FljB phase 2 flagellin and enables secretion of the unassembled FliC180 protein for maximal interaction with TLR5. It should be noted that it is flagellin and not assembled flagella that interacts with TLR5 to activate NF-kB. We thus selected use of the ΔfliC180 and Δ(hin-fljBA)-219 mutations in the final SDAAS strains. Characterization of strains for synthesis and secretion of the FliC180 flagellin used cell fractionation studies with use of antisera against different segments of the FliC protein for western blotting in addition to motility assays as described in WO 2020/096994 A1 and PCT/US21/30077.

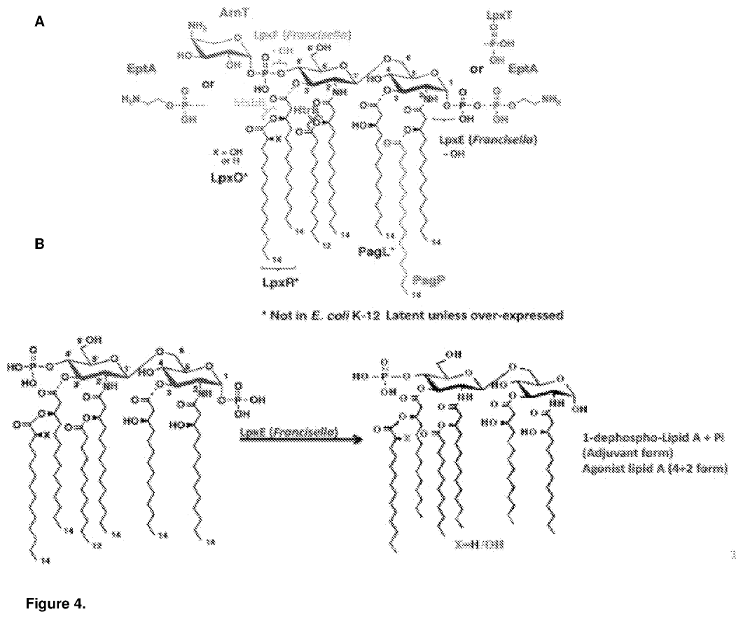

Many other SDAAS strain modifications evaluated concerned the structure of LPS lipid A (FIG. 4). Initially the ΔpagP81::P_{Ipp} IpxE deletion-insertion mutation that causes Salmonella to synthesize the adjuvant mono-phosphoryl lipid A due to the codon-optimized expression of the Francisella tularensis IpxE gene (142) was included. We then demonstrated that the successive addition of the ΔpagL7, ΔIpxR9, ΔeptA4 and ΔarnT6 mutations each further enhanced activation of NF-κB synthesis in HEK293 cells displaying TLR4. We also found using the limulus test (143, 144) that the ΔeptA4, ΔarnT6 and ΔIpxR9 mutations individually and especially collectively reduced the toxicity of lipid A to essentially zero. These modifications ensure safety and greatly reduce, if not eliminate, the induction of sepsis due to the lytic release of LPS endotoxin. The choice of these mutations to include in a SDAAS strain depends, in part, on the animal host since there is a large variation in the sensitivity of different animal species to the toxicity of the LPS lipid A endotoxin. Thus, while poultry are quite resistant/tolerant of lipid A toxicity, humans and especially horses are very susceptible to lipid A induced septic shock. We therefore contain various combinations of these mutations (ΔpagP8 or ΔpagP81::P_{Ipp} IpxE, ΔpagL7, ΔIpxR9, ΔarnT6 and ΔeptA4) in SDAAS strains to be investigated in the studies described below.

Since absence of LPS O-antigen accelerates lysis of SDAAS strains, enhances invasion into macrophages and other cell types, makes strains sensitive to complement and defensins and thus significantly reduces virulence, we investigated inclusion of the ΔwaaC41, ΔwaaG42, ΔwaaL46, Δrfc-48, ΔwbaP45 and/or Δpmi-2426 mutations for their individual effects on synthesis and secretion of flagellin, motility, interaction with HEK cells, effects on endotoxicity, sensitivity to bile salts and polymyxin and invasion into INT-407 and RAW264.7 cells. In comparative studies, the Family A strains χ12555 (ΔwaaC41) and χ12556 (ΔwaaG42) with shortened LPS core structures were highly invasive and superior in activation of TLR4 in comparison to their parent χ12515, and their sib χ12557 (ΔwaaL46 with a complete LPS core but lacking the LPS O-antigen), were defective in secreting FliC180 and not effective in activating TLR5. These results were confirmed in studies with other Family A strains χ12558, χ12559 and χ12560 derived from χ12517 (see Table 5) and the Family B strains χ12504 (parent) and derivatives χ12545 (ΔwaaC41), χ12546 (ΔwaaG42) and χ12549 (ΔwaaL46) (Table 6). Since the ability to activate TLR5 was deemed to be highly desirable, we commenced a study of how best to truncate the LPS O-antigen since lack of O-antigen did enhance invasion and activation of TLR4 in comparison to the parent or wild-type S. typhimurium strains. We therefore comparatively evaluated Family B strains with ΔwaaL46, ΔwbaP45, Δpmi-2426 and Δrfc-48 mutations (χ12641, χ12626, χ12620 and χ12621 derived from χ12612) (see Table 6) for their individual effects on synthesis and secretion of flagellin, motility, and interaction with HEK cells displaying TLR4 and TLR5. Although the results were not significantly different, on average the results with χ12626 with the ΔwbaP45 mutation were judged to be best and selection of this mutation enables continued synthesis if the WaaL enzyme which could be used in some future SDAAS strain construction to display carbohydrate antigens that might also enhance recruitment of innate immunity.

Since recruitment of innate responses due to interaction with internal TLR and NOD receptors are enhanced by lysis of SDAAS cells in the cytosol of infected cells, we included the ΔsifA26 mutation that enables Salmonella to escape from the Salmonella containing vesicle (SCV) to enter the cytosol of infected cells (145) into multiple Family A and B strains (Tables 5 and 6) including the Family A strain χ12650 and the Family B strain χ12626. In this regard, the Family A strain χ12650 was superior in recruiting innate immune responses in HEK cells with TLR8 (responsive to ssRNA), TLR9 (responsive to CpG sequences in DNA), NOD1 (responsive to peptidoglycan-derived muropeptides containing DAP) and NOD2 (responsive to ssRNA and muramyl dipeptides) (see WO 2020/096994 A1 and PCT/US21/30077) since Family A strains quickly commence to lyse after invasion into the HEK cells. On the other hand, Family B strains cannot be tested in this way since they require several cell divisions to commence to lyse that would require some 30 to 50 h in situ and these assays with HEK cells are monitored for only up to 24 h. It should be noted that the S. typhimurium ΔsifA26 mutant is totally attenuated after oral inoculation of mice yet colonizes the spleen to almost the same high titer as its wild-type parent. A similar result was observed when this ΔsifA26 mutant was orally administered to day-of-hatch chicks.

The last modification being evaluated is the inclusion of the ΔrecA62 mutation that prevents viable recombination events since recombination leads to fragmentation of the genome such that recombination is lethal (Willetts et al. 1969). This is expected to enhance the release of CpG-containing DNA fragments upon cell lysis and thus heightened activation of TLR9. Since further genetic modification is not possible after introducing a recA mutation, the construction of the Family A strain χ12661 (which improved TLR9 activation in HEK cells) and the Family B strain χ12669 were the last modifications made to SDAAS strains (Tables 5 and 6). S. typhimurium with the ΔrecA62 mutation is totally avirulent in mice and when orally administered to day-of-hatch chicks. Although addition of the ΔrecA62 mutation might be a beneficial last step, such strains have an increased generation time since about 10 percent of cells die each cell division cycle so that achieving high-titer yields of such strains is delayed adding slightly to the cost of manufacture.

The objective of our efforts is to develop safe, efficacious SDAAS strains for in ovo inoculation into 18-day old chicken embryos to induce innate immune responses to decrease the ability of a diversity of enteric pathogens to infect and colonize newly hatched chicks. However, since newly hatched chicks are quite resistant to display of invasive disease by S. typhimurium and especially its mutants, we have investigated safety of SDAAS strains administered by various mucosal and parenteral route to 6 to 8 week-old BALB/c mice. We therefore initially evaluated the relative attenuation/virulence of the Family A strain χ12517 and the Family B strain χ12518 by delivery of doses of 10⁴, 10⁵, 10⁶ and 10⁷ CFU by the i.v. route, doses of 10⁵, 10⁶, 10⁷ and 10⁸ CFU by the i.n. and s.c. routes, and 10⁹ CFU by the oral route. All mice survived challenge at all doses by all routes when infected with the Family A strain χ12517. However, some mice died when infected with the Family B strain χ12518 at doses above 10⁶ CFU by the i.v. and i.n. routes and above 10⁷ CFU by the s.c. route, while all mice survived oral inoculation. These results indicated that the several cell divisions in vivo of the Family B strains, due to the regulated delayed lysis in vivo attribute would necessitate animal studies using lower doses or the need to introduce additional mutations to preclude excess inflammatory responses leading to mortality (either by increasing attenuation and/or decreasing the number of cell divisions prior to lysis). We therefore evaluated the virulence of the Family B stains χ12612, χ12621 and χ12626 (Table 6). While χ12612 was less virulent than χ12518 by all routes, further desired attenuation was associated with the loss of O-antigen synthesis in χ12621 and χ12626. χ12621 and χ12626 were thus tolerated at higher doses by the i.v., s.c. and i.m. routes and were fully tolerated at the highest doses tested by the mucosal i.n. and oral routes. With regard to Family A strains, χ12650 and its ΔrecA62 derivative χ12661 were completely safe at oral and intranasal routes of administration to BALB/c mice at doses of 1.4×10⁹ CFU. In another study to evaluate delivery of χ12650 and χ12661 by different routes to 6- to 8-week-old BALB/c mice to examine induction of cytokine synthesis, both strains were fully safe with no adverse symptoms when administered orally at 1.4-2.0×10⁹ CFU, intranasally at 1.4-2.0×10⁹ CFU and intravenously at 3.5-5.0×10⁷ CFU.

Based on results of other studies in improving PIESV vector strains for synthesis and delivery of protective antigens to induce protective immunity against various bacterial, viral and parasite pathogens, we have proven the efficacy of using rhamnose-regulated genes to complement using arabinose-regulated genes. Since making SDAAS strains dependent on two sugars for viability will enhance their safety, we have constructed the suicide vectors ΔP_{asdA}::TT rhaRS P_{rhaBAD} asd, ΔP_{dadB}::TT rhaRS P_{rhaBAD} dadB and ΔP_{murA}::TT rhaRS P_{rhaBAD} murA and are using them to construct a series of Family B strains derived from χ12612 and χ12626 (Table 6). We have thus constructed χ12648 from χ12612 and χ12649 from χ12626 with the ΔP_{dadB}::TT rhaRS P_{rhaBAD1} dadB mutation (Table 6).

### Example 4. Ability of Co-Administration of SDAAS Strain with BCG with and without Co-Administration of a PIESV Delivering Multiple M. tuberculosis (Mtb) Protective Antigens to Confer Protection to Mice Subsequently Challenged with M. tuberculosis H37Rv

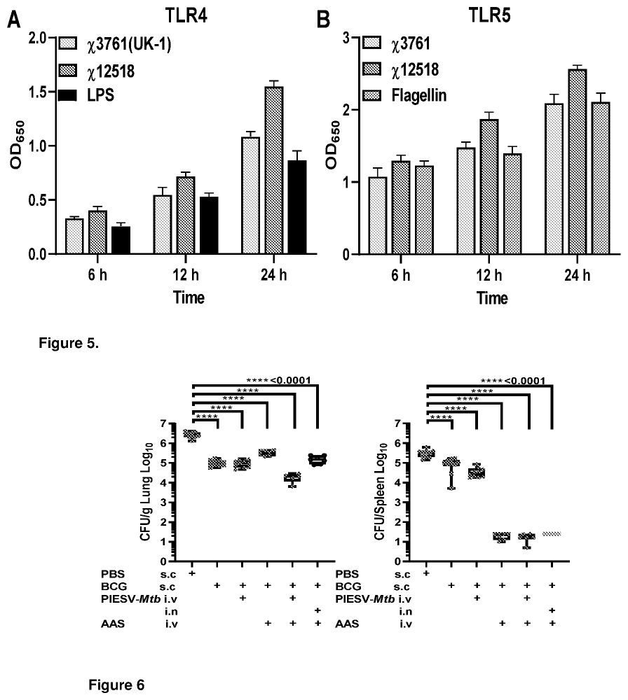

We used several different early version prospective SDAAS strains to administer by s.c., oral and i.v. routes to mice along with s.c. immunization with 5×10⁴ CFU of BCG (ATCC 35734) without and with immunization with the PIESV strain χ12068 (pYA4891 encoding ESAT6-CFP10-Ag85A) (112) (ΔP_{murA25}::TT araC P_{araBAD} murA ΔasdA27::TT araC P_{araBAD} c2 Δ(wza-wcaM)-8 Δpmi-2426 ΔrelA197::araC P_{araBAD} IacI TT ΔrecF126 ΔsifA26 ΔwaaL46 ΔpagL 19::TT araC P_{araBAD} waaL) administered orally at a dose of 10⁹ CFU or i.n. at a dose of 10⁷ CFU or i.v. at a dose of 5×10⁴ CFU. The results of one such study using the SDAAS strain χ12518 (Δalr-3 ΔP_{dadB66}::TT araC P_{araBAD} dadB ΔP_{asdA55}::TT araC P_{araBAD} asdA ΔfliC180 ΔpagP81::P_{Ipp} IpxE ΔpagL7 ΔIpxR9), which has enhanced abilities to activate TLR4 and TLR5 (FIG. 5), are presented in FIG. 6. This is the first time any vaccination regimen has resulted in undetectable Mtb CFU in spleens by any immunization protocol 35 days after receiving the virulent M. tuberculosis H37Rv challenge dose. Strikingly, this was observed in the mice just receiving the SDAAS strain with BCG. These results more than justify further development and validation of using SDAAS strains to augment induction of protective immunity by a diversity of vaccines in different animal hosts. In further analyses of these studies (see WO 2020/096994 A1 and PCT/US21/30077), it was observed that the co-administration of the SDAAS Family B strain χ12518 also augmented levels of antibodies to Mtb antigens as well as antigen-specific cellular immune responses.

### Example 5. Safety of Administration of SDAAS Strains to Embryonated Chicken Eggs at 18 Days of Incubation

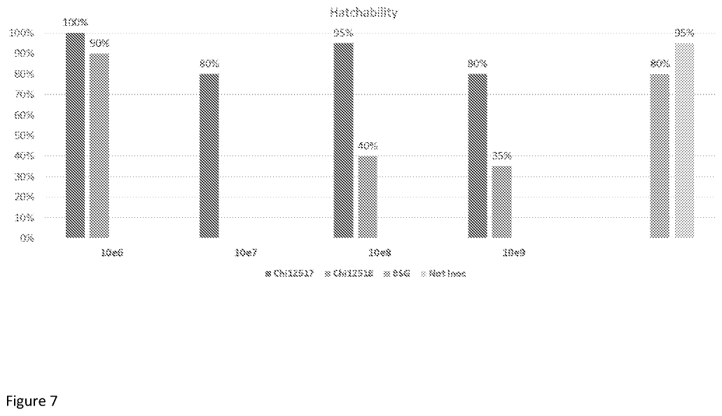

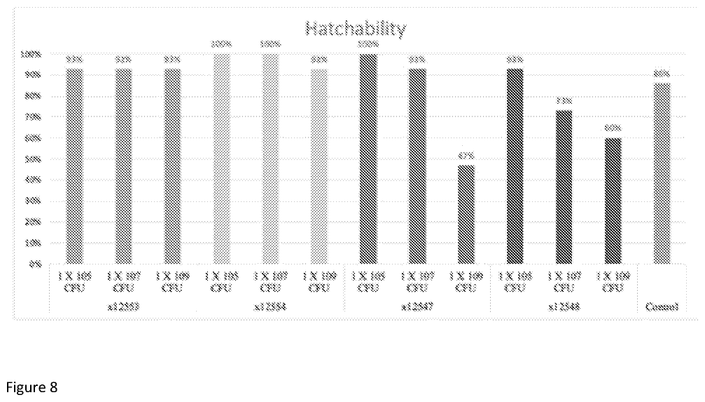

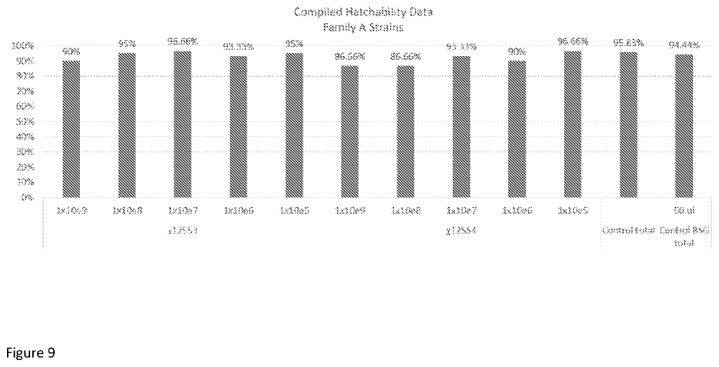

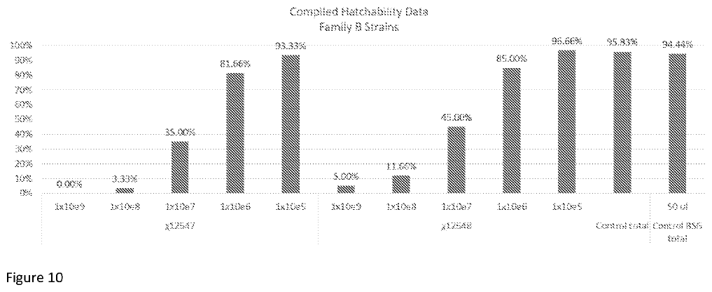

In preliminary studies (FIG. 7) with the Family A strain χ12517 and the Family B strain χ12518 we observed high hatchability with χ12517 at doses of 1×10⁸ CFU and for χ12518 at doses of 1×10⁶ CFU when inoculated into 18-day old embryos. We also more thoroughly evaluated derivatives with the added Δ(hin-fljBA)-219 mutation to enhance secretion of the FliC180 flagellin. The results (FIG. 8) showed that administration of SDAAS strains (described in Tables 3, 5 and 6) to embryonated chicken eggs at 18 days of incubation is safe and does not interfere with hatchability depending on the strain and dose inoculated. In this study, we delivered three different doses (1×10⁵ CFU, 1×10⁷ CFU and 1X×10⁹ CFU) of strains χ12553, χ12554, χ12547 and χ12548 in the amniotic fluid of white leghorn embryos and also included a control group that was not inoculated and had an intact shell. Family A strains χ12553 (Δalr-3 ΔdadB4 ΔasdA33 ΔfliC180 Δ(hin-fljBA)-219) and χ12554 (same as χ12553 plus ΔpagP81::P_{Ipp} IpxE ΔpagL7 ΔIpxR9) undergo lysis immediately after administration and hatchability was very good and independent of dose. Family B strains χ12547 (Δalr-3 ΔP_{dadB66}::TT araC P_{araBAD} dadB ΔP_{asdA55}::TT araC P_{araBAD} asdA ΔfliC180 Δ(hin-fljBA)-219) and χ12548 (χ12547 plus ΔpagP81::P_{Ipp} IpxE ΔpagL7 ΔIpxR9) that are capable of 2 to 4 cell divisions before lysis caused decreased hatchability with increasing doses. These studies were repeated multiple times and the composite results of these studies are displayed in FIGS. 9 and 10. In these experiments, 20 eggs were inoculated per strain per dose. From the chicks that hatched, 5 newly hatched chicks were euthanized and necropsied to collect organ samples to detect SDAAS strains. Organs collected were spleen, liver and lungs. Samples were homogenized individually. The tissue homogenates were plated on LB agar plate with and without D-alanine, DAP and arabinose. All chicks from in ovo inoculation of 18-day embryos with Family A SDAAS strains χ12553 and χ12554 were totally devoid of viable colony-forming cells of χ12553 and χ12554 whereas all tissues from all chicks hatched after in ovo inoculation with the Family B SDAAS strains χ12547 and χ12548 gave rise to colonies on the LB agar plates supplemented with D-alanine, DAP and arabinose but yielded no colonies when tissue extracts were streaked on LB agar plates devoid of supplements.

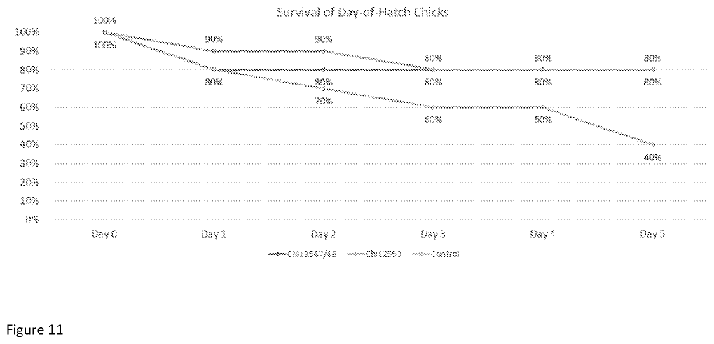

Based on these studies, it appears that the mutations ΔpagP81::P_{Ipp} IpxE, ΔpagL7 and ΔIpxR9 in strains χ12554 and χ12548 enhance safety (tolerability) in 18-day old chick embryos. In support of this, we had compared a series of Family A strains in which we introduced the ΔpagP8 mutation into χ12553 to yield χ12606 (to compare with the χ12554 derivatives that had the ΔpagP81::P_{Ipp} IpxE mutation to cause synthesis of the adjuvant form of lipid A, MPLA). The data on hatchability of 18-day old inoculated chick embryos presented in FIG. 11 reveals that the ΔpagP81::P_{Ipp} IpxE mutation is more beneficial than the ΔpagP8 mutation even though inclusion of other mutations modifying lipid A along with the ΔsifA26 mutation in χ12650 contribute to improved hatchability. Based on these studies, we are improving the Family A strain χ12650 by replacing the ΔpagP8 mutation with the ΔpagP81::P_{Ipp} IpxE mutation after which we will add the ΔrecA62 mutation. In regard to Family B strains, we believe that χ12548 could be rendered safer and more efficacious by addition of mutations such as ΔarnT6, ΔeptA4, ΔwbaP45, ΔsifA26 and ΔrecA62 that have been important further modifications of SDAAS strains used for mice (see above and Table 6). These newly constructed Family B strains χ12612, χ12626, χ12668 and χ12669 are currently being evaluated for efficacy in 18-day old chick embryos.

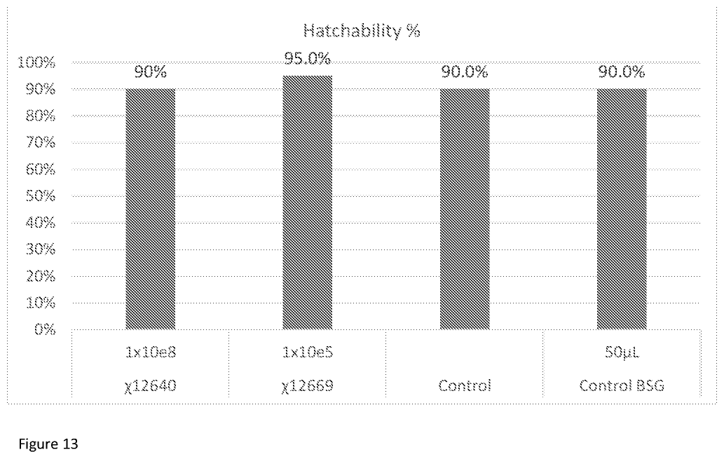

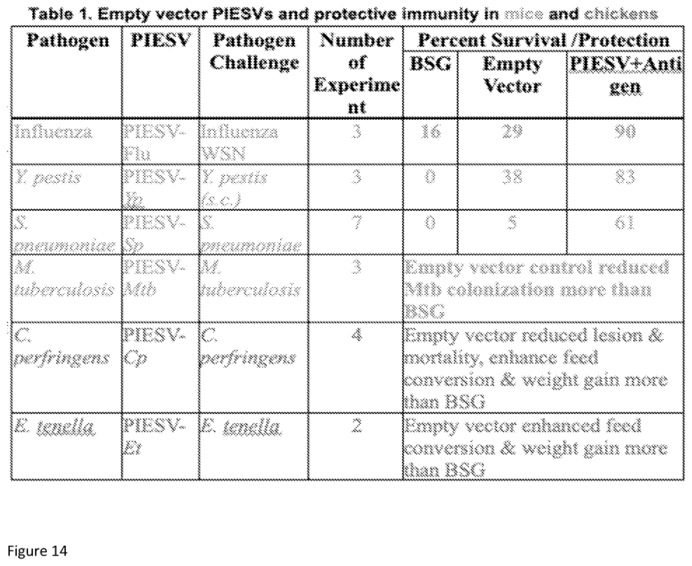

In this regard, FIG. 13 presents data demonstrating safety and high hatchability of 18-day old chick embryos inoculated with either 1×10⁸ CFU of Family A strain 112640 (Δalr-3 ΔdadB4 ΔasdA33 ΔfliC180 Δ(hin-fljBA)-219 ΔpagP8 ΔIpxR9 ΔpagL7ΔeptA4 ΔarnT6) or 1×10⁵ CFU of Family B strain χ12669 (ΔP_{asdA55}::TT araC P_{araBAD} asd Δalr-3 ΔP_{dadB66}::TT araC P_{araBAD} dadB ΔfliC180 ΔpagP81::P_{Ipp} IpxE ΔpagL7 ΔIpxR9 Δ(hin-fljBA)-219 ΔarnT6 ΔeptA4 ΔsifA26 ΔwbaP45 ΔrecA62). The positive results with the Family B strain are now being confirmed with testing of administering higher doses (1×10⁶ CFU and above) and determining viable titers in newly hatched chicks as a function of age. We postulate that inclusion of the ΔrecA62 mutation is contributing to the success with the χ12669 strain.

### Example 6. Construction of New Family a and Family B SDAAS Strains

Using suicide vectors to introduce mutations described in Table 2, various derivative SDAAS strains were constructed that would be enhanced in stimulating innate immunity by interaction with TLR4, TLR5, TLR8, TLR9, NOD1 and NOD2 and have improved safety in not reducing hatchability of in ovo inoculated embryonated eggs. These strains are listed in Tables 5 and 6 and a description of their construction is described in Example 3 above.

### Example 7. Evaluation of Protection Against Challenge of Day-of-Hatch Chicks with an O78 APEC Strain Afforded by Inoculation of 18-Day Old Chick Embryos with SDAAS Strains

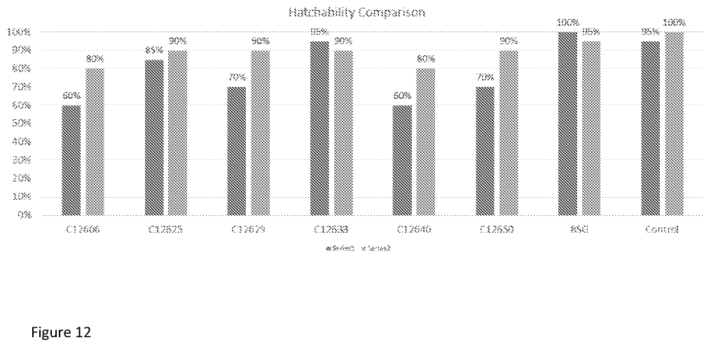

The O78 serotype APEC strain χ7122 (150) is a highly virulent strain causing air sacculitis, colisepticemia and high mortality in chickens. In an experiment evaluating hatchability of 18-day old chick embryos with χ12553, χ12554, χ12547 and χ12548, we challenged healthy hatched chicks by inoculation with 2×10⁷ CFU of χ7122 injected into the yolk sacs within their abdomen. The results of this study presented in FIG. 12 reveal that the Family A and B SDAAS strains both afford protection to this lethal APEC challenge. It is unfortunate that the study was not continued beyond 5 days since it is likely that the chicks challenged from non-SDAAS inoculated embryos would continue to develop disease symptoms and display mortality. It should also be noted that the APEC challenge doses used were higher than needed to cause infection and colonization, which would generally be by a mucosal route (aerosol due to scratching and orally due to consumption). Although preliminary in nature, these results indicate that administration of SDAAS strains to 18-day old chick embryos is safe and effective in protecting newly hatched chicks from infection and disease by an avian enteric pathogen. These results thus mirror results of administering SDAAS strains to mice to enhance induction of immune responses and protective immunity. This, of course, amplifies the generality of the beneficial contribution of the adjuvant potential of live attenuated Salmonella strains as revealed by the collective data presented in Table 1 with four studies in mice and two in chickens (vaccines against Eimeria tenella and Clostridium perfringens).

Following modification of the Family A strain χ12650 and the newly constructed Family B strains χ12612 and χ12626 currently in progress as described above including the potential substitution of some arabinose-regulated gene for a rhamnose-regulated gene in the Family B lineage (see χ12648 and χ12648 in Table 6), we will thoroughly validate safety to enable high hatchability of in ovo inoculated 18-day old embryos. This will be followed by continued studies to demonstrate protection against APEC infections and commencement of studies on inhibition of infection, colonization and disease by exposure of newly hatched chicks to oral infections with Salmonella spp, Campylobacter jejuni, Clostridium perfringens, and Listeria monocytogenes. Such studies are directed at reducing disease and colonization such that these human enteropathogens will be less likely to be transmitted through the food chain to humans thus enhancing food safety. In addition, there should be a reduced need in the poultry industry to control bacterial pathogen populations by use of antibiotics. This in turn should reduce selection for antibiotic-resistant bacteria that can also be transmitted through the food chain to humans.

### RELATED REFERENCES

Sanapala, S., L. Mosca, S. Wang, and R. Curtiss. 2018. Comparative evaluation of Salmonella typhimurium vaccines derived from UK-1 and 14028S: Importance of inherant virulence. PloS One September 7; 13 (9):e0203526. doi: 10.1371/journal.pone.0203526. eCollection 2018.PMID: 30192849 Genetic location of certain mutations conferring recombination deficiency in Escherichia coli.
Willetts N S, Clark A J, Low B. J Bacteriol. 1969 January; 97(1):244-9. doi: 10.1128/jb.97.1.244-249.1969. PMID: 4884815 Free PMC article.
1. MCKENNA M. The Surprising Origin of Chicken as a Dietary Staple. National Geographic 2018.
2. Boyd W. Making Meat: Science, Technology, and American Poultry Production. Technology and Culture: The Johns Hopkins University Press; 2001. p. 631-64.
3. Humphrey T. Are happy chickens safer chickens? Poultry welfare and disease susceptibility. Br Poult Sci. 2006; 47(4):379-91. doi: 10.1080/00071660600829084. PubMed PMID: 16905463.
4. Manyi-Loh C, Mamphweli S, Meyer E, Okoh A. Antibiotic Use in Agriculture and Its Consequential Resistance in Environmental Sources: Potential Public Health Implications. Molecules. 2018; 23 (4). Epub 2018/03/30. doi: 10.3390/molecules23040795. PubMed PMID: 29601469; PubMed Central PMCID: PMCPMC6017557.
5. Meek R W, Vyas H, Piddock L J. Nonmedical Uses of Antibiotics: Time to Restrict Their Use? PLoS Biol. 2015; 13 (10):e1002266. Epub 2015/10/07. doi: 10.1371/journal.pbio.1002266. PubMed PMID: 26444324; PubMed Central PMCID: PMCPMC4621705.
6. Mehdi Y, Létourneau-Montminy M P, Gaucher M L, Chorfi Y, Suresh G, Rouissi T, et al. Use of antibiotics in broiler production: Global impacts and alternatives. Anim Nutr. 2018; 4(2):170-8. Epub 2018/04/03. doi: 10.1016/j.aninu.2018.03.002. PubMed PMID: 30140756; PubMed Central PMCID: PMCPMC6103476.
7. Muaz K, Riaz M, Akhtar S, Park S, Ismail A. Antibiotic Residues in Chicken Meat: Global Prevalence, Threats, and Decontamination Strategies: A Review. J Food Prot. 2018; 81(4):619-27. doi: 10.4315/0362-028X.JFP-17-086. PubMed PMID: 29537307.
8. Roth N, Käsbohrer A, Mayrhofer S, Zitz U, Hofacre C, Domig K J. The application of antibiotics in broiler production and the resulting antibiotic resistance in Escherichia coli: A global overview. Poult Sci. 2019; 98(4):1791-804. doi: 10.3382/ps/pey539. PubMed PMID: 30544256; PubMed Central PMCID: PMCPMC6414035.
9. Chai S J, Cole D, Nisler A, Mahon B E. Poultry: the most common food in outbreaks with known pathogens, United States, 1998-2012. Epidemiol Infect. 2017; 145(2):316-25. Epub 2016/10/26. doi: 10.1017/S0950268816002375. PubMed PMID: 27780481.
10. Dewey-Mattia D, Manikonda K, Hall A J, Wise M E, Crowe S J. Surveillance for Foodborne Disease Outbreaks—United States, 2009-2015. MMWR Surveill Summ. 2018; 67(10):1-11. Epub 2018/07/27. doi: 10.15585/mmwr.ss6710a1. PubMed PMID: 30048426; PubMed Central PMCID: PMCPMC6061962.
11. Cunha MPV, Saidenberg A B, Moreno A M, Ferreira A J P, Vieira M A M, Gomes T A T, et al. Pandemic extra-intestinal pathogenic Escherichia coli (ExPEC) clonal group 06-B2-ST73 as a cause of avian colibacillosis in Brazil. PLoS One. 2017; 12 (6):e0178970. Epub 2017/06/08. doi: 10.1371/journal.pone.0178970. PubMed PMID: 28594893; PubMed Central PMCID: PMCPMC5464619.
12. LeStrange K, Markland S M, Hoover D G, Sharma M, Kniel K E. An evaluation of the virulence and adherence properties of avian pathogenic. One Health. 2017; 4:22-6. Epub 2017/08/21. doi: 10.1016/j.onehlt.2017.08.001. PubMed PMID: 28924584; PubMed Central PMCID: PMCPMC5591385.
13. Maluta R P, Logue C M, Casas M R, Meng T, Guastalli E A, Rojas T C, et al.
Overlapped sequence types (STs) and serogroups of avian pathogenic (APEC) and human extra-intestinal pathogenic (ExPEC) Escherichia coli isolated in Brazil. PLoS One. 2014; 9 (8):e105016. Epub 2014/08/12. doi: 10.1371/journal.pone.0105016. PubMed PMID: 25115913; PubMed Central PMCID: PMCPMC4130637.
14. Logue C M, Wannemuehler Y, Nicholson B A, Doetkott C, Barbieri N L, Nolan L K. Comparative Analysis of Phylogenetic Assignment of Human and Avian ExPEC and Fecal Commensal. Front Microbiol. 2017; 8:283. Epub 2017/02/23. doi: 10.3389/fmicb.2017.00283. PubMed PMID: 28280491; PubMed Central PMCID: PMCPMC5322314.
15. Sarowska J, Futoma-Koloch B, Jama-Kmiecik A, Frej-Madrzak M, Ksiazczyk M, Bugla-Ploskonska G, et al. Virulence factors, prevalence and potential transmission of extraintestinal pathogenic. Gut Pathog. 2019; 11:10. Epub 2019/02/21. doi: 10.1186/s13099-019-0290-0. PubMed PMID: 30828388; PubMed Central PMCID: PMCPMC6383261.
16. Johnson J R, Delavari P, O'Bryan T T, Smith K E, Tatini S. Contamination of retail foods, particularly turkey, from community markets (Minnesota, 1999-2000) with antimicrobial-resistant and extraintestinal pathogenic Escherichia coli. Foodborne Pathog Dis. 2005; 2(1):38-49. doi: 10.1089/fpd.2005.2.38. PubMed PMID: 15992297.
17. Giufrè M, Graziani C, Accogli M, Cerquetti M. Food reservoir for Escherichia coli causing urinary tract infections. Emerg Infect Dis. 2010; 16(6):1048-9; author reply 9. doi: 10.3201/eid1606.100158. PubMed PMID: 20507779; PubMed Central PMCID: PMCPMC3086230.
18. Andrews-Polymenis H L, Bäumler A J, McCormick B A, Fang F C. Taming the elephant: Salmonella biology, pathogenesis, and prevention. Infect Immun. 2010; 78(6):2356-69. Epub 2010/04/12. doi: 10.1128/IAI.00096-10. PubMed PMID: 20385760; PubMed Central PMCID: PMCPMC2876576.
19. Scallan E, Hoekstra R M, Angulo F J, Tauxe R V, Widdowson M A, Roy S L, et al. Foodborne illness acquired in the United States—major pathogens. Emerg Infect Dis. 2011; 17(1):7-15. doi: 10.3201/eid1701.p 11101. PubMed PMID: 21192848; PubMed Central PMCID: PMCPMC3375761.
20. Hoffmann S, Batz M B, Morris J G. Annual cost of illness and quality-adjusted life year losses in the United States due to 14 foodborne pathogens. J Food Prot. 2012; 75(7):1292-302. doi: 10.4315/0362-028X.JFP-11-417. PubMed PMID: 22980013.
21. Schleifer J H, Juven B J, Beard C W, Cox N A. The susceptibility of chicks to Salmonella montevideo in artificially contaminated poultry feed. Avian Dis. 1984; 28(2):497-503. PubMed PMID: 6743181.
22. Williams J E, Benson S T. Survival of Salmonella typhimurium in poultry feed and litter at three temperatures. Avian Dis. 1978; 22(4):742-7. PubMed PMID: 373743.
23. (CDC) CfDCaP. Salmonella serotype Montevideo infections associated with chicks—Idaho, Washington, and Oregon, spring 1995 and 1996. MMWR Morb Mortal Wkly Rep. 1997; 46(11):237-9. PubMed PMID: 9082177.
24. Batz M B, Doyle M P, Morris G, Painter J, Singh R, Tauxe R V, et al. Attributing illness to food. Emerg Infect Dis. 2005; 11(7):993-9. doi: 10.3201/eid1107.040634. PubMed PMID: 16022770; PubMed Central PMCID: PMCPMC3371809.
25. Batz M B, Hoffmann S, Morris J G. Ranking the disease burden of 14 pathogens in food sources in the United States using attribution data from outbreak investigations and expert elicitation. J Food Prot. 2012; 75(7):1278-91. doi: 10.4315/0362-028X.JFP-11-418. PubMed PMID: 22980012.
26. Corrente M, Sangiorgio G, Grandolfo E, Bodnar L, Catella C, Trotta A, et al. Risk for zoonotic Salmonella transmission from pet reptiles: A survey on knowledge, attitudes and practices of reptile-owners related to reptile husbandry. Prev Vet Med. 2017; 146:73-8. Epub 2017/07/25. doi: 10.1016/j.prevetmed.2017.07.014. PubMed PMID: 28992930.
27. Gantois I, Ducatelle R, Pasmans F, Haesebrouck F, Gast R, Humphrey T J, et al. Mechanisms of egg contamination by Salmonella Enteritidis. FEMS Microbiol Rev. 2009; 33(4):718-38. Epub 2009/01/21. doi: 10.1111/j.1574-6976.2008.00161.x. PubMed PMID: 19207743.
28. (CDC) CfDCaP. Multistate outbreaks of Salmonella infections associated with live poultry—United States, 2007. MMWR Morb Mortal Wkly Rep. 2009; 58(2):25-9. PubMed PMID: 19165136.
29. (CDC) CfDCaP. Three outbreaks of salmonellosis associated with baby poultry from three hatcheries—United States, 2006. MMWR Morb Mortal Wkly Rep. 2007; 56(12):273-6. PubMed PMID: 17392678.
30. Altekruse S F, Bauer N, Chanlongbutra A, DeSagun R, Naugle A, Schlosser W, et al. Salmonella enteritidis in broiler chickens, United States, 2000-2005. Emerg Infect Dis. 2006; 12(12):1848-52. doi: 10.3201/eid1212.060653. PubMed PMID: 17326935; PubMed Central PMCID: PMCPMC3291361.
31. Patrick M E, Adcock P M, Gomez T M, Altekruse S F, Holland B H, Tauxe R V, et al. Salmonella enteritidis infections, United States, 1985-1999. Emerg Infect Dis. 2004; 10(1):1-7. doi: 10.3201/eid1001.020572. PubMed PMID: 15078589; PubMed Central PMCID: PMCPMC3322758.
32. Linam W M, Gerber M A. Changing epidemiology and prevention of Salmonella infections. Pediatr Infect Dis J. 2007; 26(8):747-8. doi: 10.1097/INF.0b013e3181376abc. PubMed PMID: 17848890.
33. Braden C R. Salmonella enterica serotype Enteritidis and eggs: a national epidemic in the United States. Clin Infect Dis. 2006; 43(4):512-7. Epub 2006/07/03. doi: 10.1086/505973. PubMed PMID: 16838242.
34. Scallan E, Mahon B E. Foodborne Diseases Active Surveillance Network (FoodNet) in 2012: a foundation for food safety in the United States. Clin Infect Dis. 2012; 54 Suppl 5:S381-4. doi: 10.1093/cid/cis257. PubMed PMID: 22572657; PubMed Central PMCID: PMCPMC3348949.
35. Adak G K, Meakins S M, Yip H, Lopman B A, O'Brien S J. Disease risks from foods, England and Wales, 1996-2000. Emerg Infect Dis. 2005; 11(3):365-72. doi: 10.3201/eid1103.040191. PubMed PMID: 15757549; PubMed Central PMCID: PMCPMC3298246.
36. Allos B M, Moore M R, Griffin P M, Tauxe R V. Surveillance for sporadic foodborne disease in the 21st century: the FoodNet perspective. Clin Infect Dis. 2004; 38 Suppl 3:S115-20. doi: 10.1086/381577. PubMed PMID: 15095179.
37. Mead P S, Slutsker L, Dietz V, McCaig L F, Bresee J S, Shapiro C, et al. Food-related illness and death in the United States. Emerg Infect Dis. 1999; 5(5):607-25. doi: 10.3201/eid0505.990502. PubMed PMID: 10511517; PubMed Central PMCID: PMCPMC2627714.
38. Nachamkin I, Allos B M, Ho T. Campylobacter species and Guillain-Barré syndrome. Clin Microbiol Rev. 1998; 11(3):555-67. PubMed PMID: 9665983; PubMed Central PMCID: PMCPMC88896.
39. Altekruse S F, Stern N J, Fields P I, Swerdlow D L. Campylobacter jejuni—an emerging foodborne pathogen. Emerg Infect Dis. 1999; 5(1):28-35. doi: 10.3201/eid0501.990104. PubMed PMID: 10081669; PubMed Central PMCID: PMCPMC2627687.
40. Doorduyn Y, Van Den Brandhof W E, Van Duynhoven Y T, Breukink B J, Wagenaar J A, Van Pelt W. Risk factors for indigenous Campylobacter jejuni and Campylobacter coli infections in The Netherlands: a case-control study. Epidemiol Infect. 2010; 138(10):1391-404. Epub 2010/03/12. doi: 10.1017/S095026881000052X. PubMed PMID: 20223048.
41. DuPont H L. The growing threat of foodborne bacterial enteropathogens of animal origin. Clin Infect Dis. 2007; 45(10):1353-61. Epub 2007/10/04. doi: 10.1086/522662. PubMed PMID: 17968835.
42. Ge B, Girard W, Zhao S, Friedman S, Gaines S A, Meng J. Genotyping of Campylobacter spp. from retail meats by pulsed-field gel electrophoresis and ribotyping. J Appl Microbiol. 2006; 100(1):175-84. doi: 10.1111/j.1365-2672.2005.02750.x. PubMed PMID: 16405698.
43. Müller P, Collins-Emerson J M, Midwinter A C, Carter P, Spencer S E, van der Logt P, et al. Molecular epidemiology of Campylobacter jejuni in a geographically isolated country with a uniquely structured poultry industry. Appl Environ Microbiol. 2010; 76(7):2145-54. Epub 2010/02/12. doi: 10.1128/AEM.00862-09. PubMed PMID: 20154115; PubMed Central PMCID: PMCPMC2849254.
44. Mullner P, Shadbolt T, Collins-Emerson J M, Midwinter A C, Spencer S E, Marshall J, et al. Molecular and spatial epidemiology of human campylobacteriosis: source association and genotype-related risk factors. Epidemiol Infect. 2010; 138(10):1372-83. Epub 2010/02/09. doi: 10.1017/S0950268809991579. PubMed PMID: 20141645.
45. Wilson D J, Gabriel E, Leatherbarrow A J, Cheesbrough J, Gee S, Bolton E, et al.
Tracing the source of campylobacteriosis. PLoS Genet. 2008; 4 (9):e1000203. Epub 2008/09/26. doi: 10.1371/journal.pgen.1000203. PubMed PMID: 18818764; PubMed Central PMCID: PMCPMC2538567.
46. Friedman C R, Hoekstra R M, Samuel M, Marcus R, Bender J, Shiferaw B, et al. Risk factors for sporadic Campylobacter infection in the United States: A case-control study in FoodNet sites. Clin Infect Dis. 2004; 38 Suppl 3:S285-96. doi: 10.1086/381598. PubMed PMID: 15095201.
47. Kaakoush N O, Castaño-Rodríguez N, Mitchell H M, Man S M. Global Epidemiology of Campylobacter Infection. Clin Microbiol Rev. 2015; 28(3):687-720. doi: 10.1128/CMR.00006-15. PubMed PMID: 26062576; PubMed Central PMCID: PMCPMC4462680.
48. Guerin M T, Sir C, Sargeant J M, Waddell L, O'Connor A M, Wills R W, et al. The change in prevalence of Campylobacter on chicken carcasses during processing: a systematic review. Poult Sci. 2010; 89(5):1070-84. doi: 10.3382/ps.2009-00213. PubMed PMID: 20371862.
49. Strachan N J, Gormley F J, Rotariu O, Ogden I D, Miller G, Dunn G M, et al.
Attribution of Campylobacter infections in northeast Scotland to specific sources by use of multilocus sequence typing. J Infect Dis. 2009; 199(8):1205-8. doi: 10.1086/597417. PubMed PMID: 19265482; PubMed Central PMCID: PMCPMC3985119.
50. Jordan K, McAuliffe O. Listeria monocytogenes in Foods. Adv Food Nutr Res.
2018; 86:181-213. Epub 2018/04/03. doi: 10.1016/bs.afnr.2018.02.006. PubMed PMID: 30077222.
51. Ranjbar R, Halaji M. Epidemiology of Listeria monocytogenes prevalence in foods, animals and human origin from Iran: a systematic review and meta-analysis. BMC Public Health. 2018; 18(1):1057. Epub 2018/08/23. doi: 10.1186/s12889-018-5966-8. PubMed PMID: 30139345; PubMed Central PMCID: PMCPMC6108140.
52. Jackson K A, Gould L H, Hunter J C, Kucerova Z, Jackson B. Listeriosis Outbreaks Associated with Soft Cheeses, United States, 1998-2014. Emerg Infect Dis. 2018; 24(6):1116-8. doi: 10.3201/eid2406.171051. PubMed PMID: 29774843; PubMed Central PMCID: PMCPMC6004860.
53. Lomonaco S, Nucera D, Filipello V. The evolution and epidemiology of Listeria monocytogenes in Europe and the United States. Infect Genet Evol. 2015; 35:172-83. Epub 2015/08/05. doi: 10.1016/j.meegid.2015.08.008. PubMed PMID: 26254574.
54. Borucki M K, Call D R. Listeria monocytogenes serotype identification by PCR. J Clin Microbiol. 2003; 41(12):5537-40. doi: 10.1128/jcm.41.12.5537-5540.2003. PubMed PMID: 14662936; PubMed Central PMCID: PMCPMC309009.
55. Lianou A, Stopforth J D, Yoon Y, Wiedmann M, Sofos J N. Growth and stress resistance variation in culture broth among Listeria monocytogenes strains of various serotypes and origins. J Food Prot. 2006; 69(11):2640-7. doi: 10.4315/0362-028x-69.11.2640. PubMed PMID: 17133807.
56. Rothrock M J, Davis M L, Locatelli A, Bodie A, McIntosh T G, Donaldson J R, et al. Occurrence in Poultry Flocks: Detection and Potential Implications. Front Vet Sci. 2017; 4:125. Epub 2017/08/11. doi: 10.3389/fvets.2017.00125. PubMed PMID: 29018807; PubMed Central PMCID: PMCPMC5615842.
57. M'Sadeq S A, Wu S, Swick R A, Choct M. Towards the control of necrotic enteritis in broiler chickens with in-feed antibiotics phasing-out worldwide. Anim Nutr. 2015; 1(1):1-11. Epub 2015/03/11. doi: 10.1016/j.aninu.2015.02.004. PubMed PMID: 29766984; PubMed Central PMCID: PMCPMC5884463.
58. Hermans P G, Morgan K L. Prevalence and associated risk factors of necrotic enteritis on broiler farms in the United Kingdom; a cross-sectional survey. Avian Pathol. 2007; 36(1):43-51. doi: 10.1080/03079450601109991. PubMed PMID: 17364509.
59. Kaldhusdal M, Benestad S L, Løvland A. Epidemiologic aspects of necrotic enteritis in broiler chickens—disease occurrence and production performance. Avian Pathol. 2016; 45(3):271-4. doi: 10.1080/03079457.2016.1163521. PubMed PMID: 26956946.
60. Timbermont L, Haesebrouck F, Ducatelle R, Van Immerseel F. Necrotic enteritis in broilers: an updated review on the pathogenesis. Avian Pathol. 2011; 40(4):341-7. doi: 10.1080/03079457.2011.590967. PubMed PMID: 21812711.
61. Dhillon A S, Roy P, Lauerman L, Schaberg D, Weber S, Bandli D, et al. High mortality in egg layers as a result of necrotic enteritis. Avian Dis. 2004; 48(3):675-80. doi: 10.1637/7113. PubMed PMID: 15529994.
62. Skinner J T, Bauer S, Young V, Pauling G, Wilson J. An economic analysis of the impact of subclinical (mild) necrotic enteritis in broiler chickens. Avian Dis. 2010; 54(4):1237-40. doi: 10.1637/9399-052110-Reg.1. PubMed PMID: 21313845.
63. Kiu R, Hall L J. An update on the human and animal enteric pathogen Clostridium perfringens. Emerg Microbes Infect. 2018; 7(1):141. Epub 2018/08/06. doi: 10.1038/s41426-018-0144-8. PubMed PMID: 30082713; PubMed Central PMCID: PMCPMC6079034.
64. Takeuchi O, Akira S. Pattern recognition receptors and inflammation. Cell. 2010; 140(6):805-20. doi: 10.1016/j.cell.2010.01.022. PubMed PMID: 20303872.
65. Lee M S, Kim Y J. Signaling pathways downstream of pattern-recognition receptors and their cross talk. Annu Rev Biochem. 2007; 76:447-80. doi: 10.1146/annurev.biochem.76.060605.122847. PubMed PMID: 17328678.
66. Suresh R, Mosser D M. Pattern recognition receptors in innate immunity, host defense, and immunopathology. Adv Physiol Educ. 2013; 37(4):284-91. doi: 10.1152/advan.00058.2013. PubMed PMID: 24292903; PubMed Central PMCID: PMCPMC4089092.
67. Dar T B, Henson R M, Shiao S L. Targeting Innate Immunity to Enhance the Efficacy of Radiation Therapy. Front Immunol. 2018; 9:3077. Epub 2019/01/14. doi: 10.3389/fimmu.2018.03077. PubMed PMID: 30692991; PubMed Central PMCID: PMCPMC6339921.
68. Wang Z, Li B, Wang S, Chen T, Ye Z. Innate Immune Cells: A Potential and Promising Cell Population for Treating Osteosarcoma. Front Immunol. 2019; 10:1114. Epub 2019/05/16. doi: 10.3389/fimmu.2019.01114. PubMed PMID: 31156651; PubMed Central PMCID: PMCPMC6531991.
69. Mifsud E J, Tan A C, Jackson D C. TLR Agonists as Modulators of the Innate Immune Response and Their Potential as Agents Against Infectious Disease. Front Immunol. 2014; 5:79. Epub 2014/03/03. doi: 10.3389/fimmu.2014.00079. PubMed PMID: 24624130; PubMed Central PMCID: PMCPMC3939722.
70. Kanzler H, Barrat F J, Hessel E M, Coffman R L. Therapeutic targeting of innate immunity with Toll-like receptor agonists and antagonists. Nat Med. 2007; 13(5):552-9. doi: 10.1038/nm1589. PubMed PMID: 17479101.
71. Baldridge J R, McGowan P, Evans J T, Cluff C, Mossman S, Johnson D, et al. Taking a Toll on human disease: Toll-like receptor 4 agonists as vaccine adjuvants and monotherapeutic agents. Expert Opin Biol Ther. 2004; 4(7):1129-38. doi: 10.1517/14712598.4.7.1129. PubMed PMID: 15268679.
72. Higgins S C, Mills K H. TLR, NLR Agonists, and Other Immune Modulators as Infectious Disease Vaccine Adjuvants. Curr Infect Dis Rep. 2010; 12(1):4-12. doi: 10.1007/s11908-009-0080-9. PubMed PMID: 21308494.
73. Wu C C, Crain B, Yao S, Sabet M, Lao F S, Tawatao R I, et al. Innate immune protection against infectious diseases by pulmonary administration of a phospholipid-conjugated TLR7 ligand. J Innate Immun. 2014; 6(3):315-24. Epub 2013/11/01. doi: 10.1159/000355217. PubMed PMID: 24192551; PubMed Central PMCID: PMCPMC3972367.
74. Verthelyi D, Gursel M, Kenney R T, Lifson J D, Liu S, Mican J, et al. CpG oligodeoxynucleotides protect normal and SIV-infected macaques from Leishmania infection. J Immunol. 2003; 170(9):4717-23. doi: 10.4049/jimmunol.170.9.4717. PubMed PMID: 12707351.
75. Allan B, Wheler C, Köster W, Sarfraz M, Potter A, Gerdts V, et al. In Ovo Administration of Innate Immune Stimulants and Protection from Early Chick Mortalities due to Yolk Sac Infection. Avian Dis. 2018; 62(3):316-21. doi: 10.1637/11840-041218-Reg.1. PubMed PMID: 30339510.
76. Abdul-Cader M S, Amarasinghe A, Palomino-Tapia V, Ahmed-Hassan H, Bakhtawar K, Nagy E, et al. In ovo CpG DNA delivery increases innate and adaptive immune cells in respiratory, gastrointestinal and immune systems post-hatch correlating with lower infectious laryngotracheitis virus infection. PLoS One. 2018; 13 (3):e0193964. Epub 2018/03/07. doi: 10.1371/journal.pone.0193964. PubMed PMID: 29513732; PubMed Central PMCID: PMCPMC5841808.
77. Kawasaki T, Kawai T. Toll-like receptor signaling pathways. Front Immunol. 2014; 5:461. Epub 2014/09/25. doi: 10.3389/fimmu.2014.00461. PubMed PMID: 25309543; PubMed Central PMCID: PMCPMC4174766.
78. Sharma J M. Resistance to Marek's Disease at Hatching in Chicken Vaccinated as Embryos with the Turkey's Herpesvirus. In: Burmester B R, editor. Avian Diseases1982. p. 134-49.
79. Peebles E D. In ovo applications in poultry: A review, Poult Sci. 2018; 97(7):2322-38. doi: 10.3382/ps/pey081. PubMed PMID: 29617899.
80. Elnesr S S, Elwan H A M, Xu Q Q, Xie C, Dong X Y, Zou X T. Effects of in ovo injection of sulfur-containing amino acids on heat shock protein 70, corticosterone hormone, antioxidant indices, and lipid profile of newly hatched broiler chicks exposed to heat stress during incubation. Poult Sci. 2019; 98(5):2290-8. doi: 10.3382/ps/pey609. PubMed PMID: 30668792.
81. Ohta Y, Kidd M T. Optimum site for in ovo amino acid injection in broiler breeder eggs. Poult Sci. 2001; 80(10):1425-9. doi: 10.1093/ps/80.10.1425. PubMed PMID: 11599700.
82. Graham B D, Selby C M, Teague K D, Graham L E, Vuong C N, Latorre J D, et al. Development of a novel in ovo challenge model for virulent Escherichia coli strains. Poult Sci. 2019; 98(11):5330-5. doi: 10.3382/ps/pez321. PubMed PMID: 31289817.
83. Roto S M, Kwon Y M, Ricke S C. Applications of In Ovo Technique for the Optimal Development of the Gastrointestinal Tract and the Potential Influence on the Establishment of Its Microbiome in Poultry. Front Vet Sci. 2016; 3:63. Epub 2016/08/17. doi: 10.3389/fvets.2016.00063. PubMed PMID: 27583251; PubMed Central PMCID: PMCPMC4987676.
84. Bailar J C, Travers K. Review of assessments of the human health risk associated with the use of antimicrobial agents in agriculture. Clin Infect Dis. 2002; 34 Suppl 3:S135-43. doi: 10.1086/340252. PubMed PMID: 11988885.
85. Cohen M L, Tauxe R V. Drug-resistant Salmonella in the United States: an epidemiologic perspective. Science. 1986; 234(4779):964-9. doi: 10.1126/science.3535069. PubMed PMID: 3535069.
86. Economou V, Gousia P. Agriculture and food animals as a source of antimicrobial-resistant bacteria. Infect Drug Resist. 2015; 8:49-61. Epub 2015/04/01. doi: 10.2147/IDR.S55778. PubMed PMID: 25878509; PubMed Central PMCID: PMCPMC4388096.
87. Sahin O, Zhang Q, Meitzler J C, Harr B S, Morishita T Y, Mohan R. Prevalence, antigenic specificity, and bactericidal activity of poultry anti-Campylobacter maternal antibodies. Appl Environ Microbiol. 2001; 67(9):3951-7. doi: 10.1128/aem.67.9.3951-3957.2001. PubMed PMID: 11525990; PubMed Central PMCID: PMCPMC93114.
88. Hassan J O, Curtiss R. Development and evaluation of an experimental vaccination program using a live avirulent Salmonella typhimurium strain to protect immunized chickens against challenge with homologous and heterologous Salmonella serotypes. Infect Immun. 1994; 62(12):5519-27. PubMed PMID: 7960134; PubMed Central PMCID: PMCPMC303297.
89. Dórea F C, Cole D J, Hofacre C, Zamperini K, Mathis D, Doyle M P, et al. Effect of Salmonella vaccination of breeder chickens on contamination of broiler chicken carcasses in integrated poultry operations. Appl Environ Microbiol. 2010; 76(23):7820-5. Epub 2010/10/01. doi: 10.1128/AEM.01320-10. PubMed PMID: 20889797; PubMed Central PMCID: PMCPMC2988591.
90. Gantois I, Ducatelle R, Timbermont L, Boyen F, Bohez L, Haesebrouck F, et al. Oral immunisation of laying hens with the live vaccine strains of TAD Salmonella vac E and TAD Salmonella vac T reduces internal egg contamination with Salmonella Enteritidis. Vaccine. 2006; 24 (37-39):6250-5. Epub 2006/06/09. doi: 10.1016/j.vaccine.2006.05.070. PubMed PMID: 16842890.
91. Cooper G L, Venables L M, Woodward M J, Hormaeche C E. Vaccination of chickens with strain CVL30, a genetically defined Salmonella enteritidis aroA live oral vaccine candidate. Infect Immun. 1994; 62(11):4747-54. PubMed PMID: 7927750; PubMed Central PMCID: PMCPMC303182.
92. Casewell M, Friis C, Marco E, McMullin P, Phillips I. The European ban on growth-promoting antibiotics and emerging consequences for human and animal health. J Antimicrob Chemother. 2003; 52(2):159-61. Epub 2003/07/01. doi: 10.1093/jac/dkg313. PubMed PMID: 12837737.
93. Pastoret P P, Jones P. Veterinary vaccines for animal and public health. Dev Biol (Basel). 2004; 119:15-29. PubMed PMID: 15742615.
94. Newell D G, Elvers K T, Dopfer D, Hansson I, Jones P, James S, et al. Biosecurity-based interventions and strategies to reduce Campylobacter spp. on poultry farms. Appl Environ Microbiol. 2011; 77(24):8605-14. Epub 2011/10/07. doi: 10.1128/AEM.01090-10. PubMed PMID: 21984249; PubMed Central PMCID: PMCPMC3233073.
95. Andres V. Biosecurity Measures to Control Salmonella and Other Infectious Agents in Pig Farms: A Review. In: Rob D, editor. comprehensive reviews in food science and food safety: Institute of Food Technologists; 2015. p. 317-35.
96. Lin J. Novel approaches for Campylobacter control in poultry. Foodborne Pathog Dis. 2009; 6(7):755-65. doi: 10.1089/fpd.2008.0247. PubMed PMID: 19425824; PubMed Central PMCID: PMCPMC3145176.
97. Hermans D, Van Deun K, Messens W, Martel A, Van Immerseel F, Haesebrouck F, et al. Campylobacter control in poultry by current intervention measures ineffective: urgent need for intensified fundamental research. Vet Microbiol. 2011; 152 (3-4):219-28. Epub 2011/03/16. doi: 10.1016/j.vetmic.2011.03.010. PubMed PMID: 21482043.
98. Doyle M P, Erickson M C. Reducing the carriage of foodborne pathogens in livestock and poultry. Poult Sci. 2006; 85(6):960-73. doi: 10.1093/ps/85.6.960. PubMed PMID: 16776463.
99. Ridley A M, Morris V K, Cawthraw S A, Ellis-Iversen J, Harris J A, Kennedy E M, et al. Longitudinal molecular epidemiological study of thermophilic campylobacters on one conventional broiler chicken farm. Appl Environ Microbiol. 2011; 77(1):98-107. Epub 2010/10/29. doi: 10.1128/AEM.01388-10. PubMed PMID: 21037294; PubMed Central PMCID: PMCPMC3019741.
100. Ridley A, Morris V, Gittins J, Cawthraw S, Harris J, Edge S, et al. Potential sources of Campylobacter infection on chicken farms: contamination and control of broiler-harvesting equipment, vehicles and personnel. J Appl Microbiol. 2011; 111(1):233-44. Epub 2011/05/20. doi: 10.1111/j.1365-2672.2011.05038.x. PubMed PMID: 21535329.
101. Arsenault J, Letellier A, Quessy S, Normand V, Boulianne M. Prevalence and risk factors for Salmonella spp. and Campylobacter spp. caecal colonization in broiler chicken and turkey flocks slaughtered in Quebec, Canada. Prev Vet Med. 2007; 81(4):250-64. Epub 2007/05/29. doi: 10.1016/j.prevetmed.2007.04.016. PubMed PMID: 17532069.
102. Näther G, Alter T, Martin A, Ellerbroek L. Analysis of risk factors for Campylobacter species infection in broiler flocks. Poult Sci. 2009; 88(6):1299-305. doi: 10.3382/ps.2008-00389. PubMed PMID: 19439643.
103. Sahin O, Kobalka P, Zhang Q. Detection and survival of Campylobacter in chicken eggs. J Appl Microbiol. 2003; 95(5):1070-9. doi: 10.1046/j.1365-2672.2003.02083.x. PubMed PMID: 14633036.
104. Tan S, Gyles C L, Wilkie B N. Evaluation of an aroA mutant Salmonella typhimurium vaccine in chickens using modified semisolid Rappaport Vassiliadis medium to monitor faecal shedding. Vet Microbiol. 1997; 54 (3-4):247-54. doi: 10.1016/s0378-1135(96)01279-5. PubMed PMID: 9100326.
105. Barrow P A. Salmonella infections: immune and non-immune protection with vaccines. Avian Pathol. 2007; 36(1):1-13. doi: 10.1080/03079450601113167. PubMed PMID: 17364505.
106. Gast R K. Serotype-specific and serotype-independent strategies for preharvest control of food-borne Salmonella in poultry. Avian Dis. 2007; 51(4):817-28. doi: 10.1637/8090-081807.1. PubMed PMID: 18251388.
107. Desin T S, Köster W, Potter A A. Salmonella vaccines in poultry: past, present and future. Expert Rev Vaccines. 2013; 12(1):87-96. doi: 10.1586/erv.12.138. PubMed PMID: 23256741.
108. Ghunaim H, Abu-Madi M A, Kariyawasam S. Advances in vaccination against avian pathogenic Escherichia coli respiratory disease: potentials and limitations. Vet Microbiol. 2014; 172 (1-2):13-22. Epub 2014/05/05. doi: 10.1016/j.vetmic.2014.04.019. PubMed PMID: 24878325.
109. Wang S, Li Y, Scarpellini G, Kong W, Shi H, Baek C H, et al. Salmonella vaccine vectors displaying delayed antigen synthesis in vivo to enhance immunogenicity. Infect Immun. 2010; 78(9):3969-80. Epub 2010/07/08. doi: IAI.00444-10 [pii]10.1128/IAI.00444-10. PubMed PMID: 20605977; PubMed Central PMCID: PMC2937466.
110. Kong W, Wanda S Y, Zhang X, Bollen W, Tinge S A, Roland K L, et al. Regulated programmed lysis of recombinant Salmonella in host tissues to release protective antigens and confer biological containment. Proc Natl Acad Sci USA. 2008; 105(27):9361-6. Epub 2008/07/07. doi: 10.1073/pnas.0803801105. PubMed PMID: 18607005; PubMed Central PMCID: PMCPMC2453710.
111. Curtiss R, 3rd, Wanda S Y, Gunn B M, Zhang X, Tinge S A, Ananthnarayan V, et al. Salmonella enterica serovar typhimurium strains with regulated delayed attenuation in vivo. Infection and immunity. 2009; 77(3):1071-82. Epub 2008/12/24. doi: 10.1128/iai.00693-08. PubMed PMID: 19103774; PubMed Central PMCID: PMCPMC2643627.
112. Juárez-Rodríguez M D, Yang J, Kader R, Alamuri P, Curtiss R, III, Clark-Curtiss J E. Live attenuated Salmonella vaccines displaying regulated delayed lysis and delayed antigen synthesis to confer protection against Mycobacterium tuberculosis. Infect Immun. 2012; 80(2):815-31. doi: 10.1128/IAI.05526-11. PubMed PMID: 22144485; PubMed Central PMCID: PMC3264310.
113. Galan J E, Nakayama K, Curtiss R, 3rd. Cloning and characterization of the asd gene of Salmonella typhimurium: use in stable maintenance of recombinant plasmids in Salmonella vaccine strains. Gene. 1990; 94(1):29-35. Epub 1990/09/28. PubMed PMID: 2227450.
114. Black S, Wright N G. Aspartic b-semialdehyde dehydrogenase and aspartic b-semialdehyde. J Biol Chem. 1955; 213(1):39-50. PubMed PMID: 14353904.
115. Nakayama K, Kelly S M, Curtiss R, III Construction of an Asd⁺ expression-cloning Vector: stable maintenance and high level expression of cloned genes in a Salmonella vaccine strain. Nat Biotech. 1988; 6(6):693-7.
116. Xin W, Wanda S Y, Zhang X, Santander J, Scarpellini G, Ellis K, et al. The Asd(+)-DadB(+) dual-plasmid system offers a novel means to deliver multiple protective antigens by a recombinant attenuated Salmonella vaccine. Infection and immunity. 2012; 80(10):3621-33. Epub 2012/08/08. doi: 10.1128/iai.00620-12. PubMed PMID: 22868499; PubMed Central PMCID: PMCPMC3457550.
117. Kong Q, Six D A, Roland K L, Liu Q, Gu L, Reynolds C M, et al. Salmonella synthesizing 1-dephosphorylated [corrected] lipopolysaccharide exhibits low endotoxic activity while retaining its immunogenicity. Journal of immunology (Baltimore, Md.: 1950). 2011; 187(1):412-23. Epub 2011/06/03. doi: 10.4049/jimmunol.1100339. PubMed PMID: 21632711; PubMed Central PMCID: PMCPMC3119770.
118. Kong Q, Six D A, Liu Q, Gu L, Wang S, Alamuri P, et al. Phosphate groups of lipid A are essential for Salmonella enterica serovar typhimurium virulence and affect innate and adaptive immunity. Infection and immunity. 2012; 80(9):3215-24. Epub 2012/07/04. doi: 10.1128/iai.00123-12. PubMed PMID: 22753374; PubMed Central PMCID: PMCPMC3418755.
119. Lee H, Hsu F F, Turk J, Groisman E A. The PmrA-regulated pmrC gene mediates phosphoethanolamine modification of lipid A and polymyxin resistance in Salmonella enterica. J Bacteriol. 2004; 186(13):4124-33. doi: 10.1128/J B.186.13.4124-4133.2004. PubMed PMID: 15205413; PubMed Central PMCID: PMCPMC421605.
120. Trent M, Raetz C. Cloning of EptA, the lipid A phosphoethanolamine transferase associated with polymyxin resistance. Journal of endotoxin research. 2002; 8:159.
121. Trent M S, Ribeiro A A, Lin S, Cotter R J, Raetz C R. An inner membrane enzyme in Salmonella and Escherichia coli that transfers 4-amino-4-deoxy-L-arabinose to lipid A: induction on polymyxin-resistant mutants and role of a novel lipid-linked donor. J Biol Chem. 2001; 276(46):43122-31. doi: 10.1074/jbc.M106961200. PubMed PMID: 11535604.
122. Liu Q, Liu Q, Yi J, Liang K, Hu B, Zhang X, et al. Outer membrane vesicles from flagellin-deficient Salmonella enterica serovar typhimurium induce cross-reactive immunity and provide cross-protection against heterologous Salmonella challenge. Scientific reports. 2016; 6:34776. Epub 2016/10/05. doi: 10.1038/srep34776. PubMed PMID: 27698383; PubMed Central PMCID: PMCPMC5048178.
123. Zieg J, Simon M. Analysis of the nucleotide sequence of an invertible controlling element. Proc Natl Acad Sci USA. 1980; 77(7):4196-200. doi: 10.1073/pnas.77.7.4196. PubMed PMID: 6933466; PubMed Central PMCID: PMCPMC349798.
124. Silverman M, Simon M. Phase variation: genetic analysis of switching mutants. Cell. 1980; 19(4):845-54. doi: 10.1016/0092-8674(80)90075-6. PubMed PMID: 6247071.
125. Silverman M, Zieg J, Simon M. Flagellar-phase variation: isolation of the rh1 gene. J Bacteriol. 1979; 137(1):517-23. PubMed PMID: 762022; PubMed Central PMCID: PMCPMC218478.
126. Kutsukake K, lino T. Inversions of specific DNA segments in flagellar phase variation of Salmonella and inversion systems of bacteriophages P1 and Mu. Proc Natl Acad Sci USA. 1980; 77(12):7338-41. doi: 10.1073/pnas.77.12.7338. PubMed PMID: 7012837; PubMed Central PMCID: PMCPMC350498.
127. Bonifield H R, Hughes K T. Flagellar phase variation in Salmonella enterica is mediated by a posttranscriptional control mechanism. J Bacteriol. 2003; 185(12):3567-74. doi: 10.1128/jb.185.12.3567-3574.2003. PubMed PMID: 12775694; PubMed Central PMCID: PMCPMC156219.
128. Kutsukake K, lino T. A trans-acting factor mediates inversion of a specific DNA segment in flagellar phase variation of Salmonella. Nature. 1980; 284(5755):479-81. doi: 10.1038/284479a0. PubMed PMID: 7360284.
129. Yamamoto S, Kutsukake K. FIjA-mediated posttranscriptional control of phase 1 flagellin expression in flagellar phase variation of Salmonella enterica serovar typhimurium. J Bacteriol. 2006; 188(3):958-67. doi: 10.1128/J B.188.3.958-967.2006. PubMed PMID: 16428400; PubMed Central PMCID: PMCPMC1347349.
130. Collins L V, Attridge S, Hackett J. Mutations at rfc or pmiattenuate Salmonella typhimurium virulence for mice. Infect Immun. 1991; 59(3):1079-85. PubMed PMID: 1997412.
131. Kong Q, Yang J, Liu Q, Alamuri P, Roland K L, Curtiss R, III. Effect of deletion of genes involved in lipopolysaccharide core and O-antigen synthesis on virulence and immunogenicity of Salmonella enterica serovar typhimurium. Infect Immun. 2011; 79(10):4227-39. Epub 2011/07/20. doi: 10.1128/IAI.05398-11. PubMed PMID: 21768282; PubMed Central PMCID: PMC3187260.
132. Stevenson G, Andrianopoulos K, Hobbs M, Reeves P R. Organization of the Escherichia coli K-12 gene cluster responsible for production of the extracellular polysaccharide colanic acid. J Bacteriol. 1996; 178(16):4885-93. PubMed PMID: 8759852; PubMed Central PMCID: PMC178271.
133. Whitfield C. Biosynthesis and assembly of capsular polysaccharides in Escherichia coli. Annual review of biochemistry. 2006; 75:39-68. doi: 10.1146/annurev.biochem.75.103004.142545. PubMed PMID: 16756484.
134. Pizarro-Cerda J, Tedin K. The bacterial signal molecule, ppGpp, regulates Salmonella virulence gene expression. Mol Microbiol. 2004; 52(6):1827-44. Epub 2004/06/10. doi: 10.1111/j.1365-2958.2004.04122.x MMI4122 [pii]. PubMed PMID: 15186428.
135. Torok I, Kari C. Accumulation of ppGpp in a relA mutant of Escherichia coli during amino acid starvation. The Journal of biological chemistry. 1980; 255(9):3838-40. Epub 1980/05/10. PubMed PMID: 6768741.
136. Xin W, Wanda S Y, Zhang X, Santander J, Scarpellini G, Ellis K, et al. The Asd⁺-DadB⁺ dual-plasmid system offers a novel means to deliver multiple protective antigens by a recombinant attenuated Salmonella vaccine. Infect Immun. 2012; 80(10):3621-33. Epub 2012/08/08. doi: 10.1128/IAI.00620-12. PubMed PMID: 22868499; PubMed Central PMCID: PMC3457550.
137. Cohen A, Laban A. Plasmidic recombination in Escherichia coli K-12: the role of recFgene function. Mol Gen Genet. 1983; 189(3):471-4. Epub 1983/01/01. PubMed PMID:6346018.
138. Kolodner R, Fishel R A, Howard M. Genetic recombination of bacterial plasmid DNA: effect of RecF pathway mutations on plasmid recombination in Escherichia coli. J Bacteriol. 1985; 163(3):1060-6. Epub 1985/09/01. PubMed PMID: 2993230; PubMed Central PMCID: PMC219238.
139. Zhang X, Wanda S Y, Brenneman K, Kong W, Roland K, Curtiss R, III. Improving Salmonella vector with rec mutation to stabilize the DNA cargoes. BMC Microbiol. 2011; 11:31. Epub 2011/02/10. doi: 1471-2180-11-31 [pii]10.1186/1471-2180-11-31. PubMed PMID: 21303535; PubMed Central PMCID: PMC3047425.
140. Beuzón C R, Méresse S, Unsworth K E, Ruiz-Albert J, Garvis S, Waterman S R, et al. Salmonella maintains the integrity of its intracellular vacuole through the action of SifA. The EMBO journal. 2000; 19(13):3235-49. doi: 10.1093/emboj/19.13.3235. PubMed PMID: 10880437; PubMed Central PMCID: PMC313946.
141. Ohlson M B, Huang Z, Alto N M, Blanc M P, Dixon J E, Chai J, et al. Structure and function of Salmonella SifA indicate that its interactions with SKIP, SseJ, and RhoA family GTPases induce endosomal tubulation. Cell host & microbe. 2008; 4(5):434-46. Epub 2008/11/11. doi: 10.1016/j.chom.2008.08.012. PubMed PMID: 18996344; PubMed Central PMCID: PMCPMC2658612.
142. Kong Q, Six D A, Roland K L, Liu Q, Gu L, Reynolds C M, et al. Salmonella synthesizing 1-dephosphorylated lipopolysaccharide exhibits low endotoxic activity while retaining its immunogenicity. J Immunol. 2011; 187(1):412-23. Epub 2011/06/03. doi: jimmunol.1100339 [pii]10.4049/jimmunol.1100339. PubMed PMID: 21632711; PubMed Central PMCID: PMC3119770.
143. Marlys Weary, John Dubczak, Jeffrey Wiggins, Joyce Bohon, Alline Brandon, Fred C. Pearson I. Validating an LAL chromogenic substrate pyrogen test for large volume parenterals. In: Watson L, and Novitsky editor. Detection of bacterial endotoxins with the Limulus amebocyte lysate test Proceedings of an international congress. 231. New York.: Alan R. Liss; 1987. p. 307-22.
144. Cooper J F. Ideal properties of a LAL reagent for pharmaceutical testing. In: Watson L, Novitsky, editors. Bacterial endotoxins Structure, biomedical significance, and detection with the Limulus amebocyte lysate test Alan R. Liss. Inc.: New York, (1985) p. 241-249: Alan R. Liss. Inc.: New York; 1985. p. 241-9.
145. Ashraf S, Kong W, Wang S, Yang J, Curtiss R, Ill. Protective cellular responses elicited by vaccination with influenza nucleoprotein delivered by a live recombinant attenuated Salmonella vaccine. Vaccine. 2011; 29(23):3990-4002. doi: 10.1016/j.vaccine.2011.03.066. PubMed PMID: 21466806; PubMed Central PMCID: PMC3092860.
146. Curtiss R, Hassan J O. Nonrecombinant and recombinant avirulent Salmonella vaccines for poultry. Vet Immunol Immunopathol. 1996; 54 (1-4):365-72. doi: 10.1016/s0165-2427(96)05683-8. PubMed PMID: 8988881.
147. Zhang X, Kelly S M, Bollen W S, Curtiss R. Characterization and immunogenicity of Salmonella typhimurium SL1344 and UK-1 delta crp and delta cdt deletion mutants. Infect Immun. 1997; 65(12):5381-7. PubMed PMID: 9393846; PubMed Central PMCID: PMCPMC175779.
148. Thompson D R, Parreira V R, Kulkarni R R, Prescott J F. Live attenuated vaccine-based control of necrotic enteritis of broiler chickens. Vet Microbiol. 2006; 113 (1-2):25-34. Epub 2005/11/11. doi: 10.1016/j.vetmic.2005.10.015. PubMed PMID: 16289639.
149. Curtiss R, Ill., S. B. Porter, M. Munson, S. A. Tinge, J. O. Hassan, C. Gentry-Weeks, et al. Nonrecombinant and recombinant avirulent Salmonella live vaccines for poultry. In: L. C. Blankenship, J. H. S. Bailey, N. A. Cox, Stern N J, Meinersmann R J, editors. Colonization control of human bacterial enteropathogens in poultry. New York: Academic Press; 1991. p. 169-98.
150. Provence D L, Curtiss R. Role of crl in avian pathogenic Escherichia coli: a knockout mutation of crl does not affect hemagglutination activity, fibronectin binding, or Curli production. Infect Immun. 1992; 60(11):4460-7. PubMed PMID: 1398960; PubMed Central PMCID: PMCPMC258189.
151. Fouts D E, Mongodin E F, Mandrell R E, Miller W G, Rasko D A, Ravel J, et al. Major structural differences and novel potential virulence mechanisms from the genomes of multiple campylobacter species. PLoS Biol. 2005; 3 (1): e15. doi: 10.1371/journal.pbio.0030015. PubMed PMID: 15660156; PubMed Central PMCID: PMC539331.
152. Parker C T, Quinones B, Miller W G, Horn S T, Mandrell R E. Comparative genomic analysis of Campylobacter jejuni strains reveals diversity due to genomic elements similar to those present in C. jejuni strain RM1221. J Clin Microbiol. 2006; 44(11):4125-35. doi: 10.1128/JCM.01231-06. PubMed PMID: 16943349; PubMed Central PMCID: PMC1698300.
153. Sambrook J, Russell D W. Molecular cloning: a laboratory manual. 3rd ed. Cold Spring Harbor, N.Y.: Cold Spring Harbor Laboratory Press; 2001.
154. Hitchcock P J, Brown T M. Morphological heterogeneity among Salmonella lipopolysaccharide chemotypes in silver-stained polyacrylamide gels. J Bacteriol. 1983; 154(1):269-77. Epub 1983/04/01. PubMed PMID: 6187729.
155. Shi H, Santander J, Brenneman K E, Wanda S Y, Wang S, Senechal P, et al. Live recombinant Salmonella Typhi vaccines constructed to investigate the role of rpoS in eliciting immunity to a heterologous antigen. PLoS One. 2010; 5 (6): e11142. Epub 2010/06/30. doi: 10.1371/journal.pone.0011142. PubMed PMID: 20585446; PubMed Central PMCID: PMC2887840.
156. Spencer W E, Christensen M J. Multiplex relative R T-PCR method for verification of differential gene expression. Biotechniques. 1999; 27(5):1044-6, 8-50, 52. doi: 10.2144/99275rr04. PubMed PMID: 10572652.
157. Naghizadeh M, Larsen F T, Wattrang E, Norup L R, Dalgaard T S. Rapid whole blood assay using flow cytometry for measuring phagocytic activity of chicken leukocytes. Vet Immunol Immunopathol. 2019; 207:53-61. Epub 2018/11/28. doi: 10.1016/j.vetimm.2018.11.014. PubMed PMID: 30593351.
158. Galen J E, Curtiss R, III. The delicate balance in genetically engineering live vaccines. Vaccine. 2014; 32(35):4376-85. doi: 10.1016/j.vaccine.2013.12.026. PubMed PMID: 24370705; PubMed Central PMCID: PMC4069233.
159. Clark-Curtiss J E, Curtiss R, III. Salmonella Vaccines: Conduits for Protective Antigens. J Immunol. 2018; 200(1):39-48. doi: 10.4049/jimmunol.1600608. PubMed PMID: 29255088.
160. Wang S, Kong Q, Curtiss R, III. New technologies in developing recombinant attenuated Salmonella vaccine vectors. Microb Pathog. 2013; 58:17-28. Epub 2012/11/13. doi: 10.1016/j.micpath.2012.10.006. PubMed PMID: 23142647; PubMed Central PMCID: PMC3587120.
161. Wang S, Li Y, Scarpellini G, Kong W, Shi H, Baek C H, et al. Salmonella vaccine vectors displaying delayed antigen synthesis in vivo to enhance immunogenicity. Infect Immun. 2010; 78(9):3969-80. Epub 2010/07/08. doi: IAI.00444-10 [pii]10.1128/IAI.00444-10. PubMed PMID: 20605977; PubMed Central PMCID: PMC2937466.
162. Wang S, Li Y, Shi H, Sun W, Roland K L, Curtiss R, III. Comparison of a regulated delayed antigen synthesis system with in vivo-inducible promoters for antigen delivery by live attenuated Salmonella vaccines. Infect Immun. 2011; 79(2):937-49. Epub 2010/12/08. doi: IAI.00445-10 [pii]10.1128/IAI.00445-10. PubMed PMID: 21134969; PubMed Central PMCID: PMC3028866.
163. Kong W, Wanda S Y, Zhang X, Bollen W, Tinge S A, Roland K L, et al. Regulated programmed lysis of recombinant Salmonella in host tissues to release protective antigens and confer biological containment. Proc Natl Acad Sci USA. 2008; 105(27):9361-6. Epub 2008/07/09. doi: 0803801105 [pii]

What is claimed is:

1. A live self-destructing attenuated adjuvant Salmonella strain, or a derivative thereof, capable of safe in ovo inoculation into embryonated avian eggs without reduction in hatchability.

2. The live self-destructing attenuated adjuvant Salmonella strain of claim 1 or derivative thereof, wherein the adjuvant comprises an attenuated Salmonella typhimurium (S. typhimurium) bacterium, comprising one or more mutations resulting in in vivo self-destruction selected from the group consisting of Δalr, ΔdadB, ΔasdA, ΔP_{asdA}::TT araC P_{araBAD} asd, ΔP_{dadB}::TT araC P_{araBAD} dadB, ΔP_{asdA}::TT rhaRS P_{rhaBAD} asd, ΔP_{dadB}::TT rhaRS P_{rhaBAD} dadB and/or ΔP_{murA}::TT rhaRS P_{rhaBAD} murA.

3. The live self-destructing attenuated adjuvant Salmonella strain of claim 2 or a derivative of, wherein the strain, or derivative thereof, further comprises mutations ΔfliC180 and Δ(hin-fljBA to enhance recruitment of innate immunity via interaction with PRR TLR5.

4. The live self-destructing attenuated adjuvant Salmonella strain of claim 2 or a derivative of, wherein the strain, or derivative thereof, further comprises ΔpagP or ΔpagP::P_{Ipp} IpxE, ΔpagL, ΔIpxR, ΔarnT and/or ΔeptA to enhance recruitment of innate immunity via interaction with PRR TLR4.

5. The live self-destructing attenuated adjuvant Salmonella strain of claim 2 or a derivative of, wherein the strain, or derivative thereof, further comprises mutations ΔwaaC, ΔwaaG, ΔwaaL, ΔwbaP, Δpmi and/or Δrfc to enable improved interaction of bacterial adjuvant surface MAMPs and DAMPs to enhance recruitment of innate immunity via interaction with host PRRs.

6. The live self-destructing attenuated adjuvant Salmonella strain of claim 2 or a derivative of, wherein the strain, or derivative thereof, further comprises mutations ΔsifA and/or ΔrecA to enhance recruitment of innate immunity via interaction with host cell internal PRRs such as, but not limited to, TLR8, TLR9, NOD1 and/or NOD2.

7. A method of inoculating embryonated avian eggs, the method comprising administering, in ovo, an effective inoculating amount of a live self-destructing attenuated adjuvant Salmonella strain, or derivative thereof, according to any of claims 1-6.

8. The method of claim 7, wherein administering induces innate immunity of hatched offspring from the inoculated embryonated avian eggs.

9. The method of claims 7 or 8, wherein administering does not reduce hatchability of the inoculated embryonated avian eggs.

10. The method of claim 7, wherein administering decreases severity of infection of hatched offspring from the inoculated embryonated avian eggs by avian pathogens.

11. The method of claim 10, wherein the avian pathogens are E. coli (APEC) strains.

12. A composition comprising an amount of a live self-destructing attenuated adjuvant Salmonella strain, or derivative thereof, according to any of claims 1-6 and a carrier.
